(19) United States
(12) Patent Application Publication
(10) Pub. No.: US 2009/0311214 A1
(43) Pub. Date: Dec. 17, 2009

(54) COMPOSITIONS AND METHODS FOR THE INNATE IMMUNE RESPONSE PATHWAY

(75) Inventors: Dean L. MANN, Rockville, MD (US); Kristina M. Harris, Annapolis, MD (US)

Correspondence Address: SUGHRUE MION, PLLC, 2100 PENNSYLVANIA AVENUE, N.W., SUITE 800, WASHINGTON, DC, 20037, US

(73) Assignee: UNIVERSITY OF MARYLAND, BALTIMORE, Baltimore, MD (US)

(21) Appl. No.: 12/482,283

(22) Filed: Jun. 10, 2009

Related U.S. Application Data
(60) Provisional application No. 61/060,783, filed on Jun. 11, 2008.

(51) Int. Cl. A61K 38/20; C12Q 1/02; A61P 29/00

(52) U.S. Cl. 424/85.2; 435/29

(57) ABSTRACT

Autoimmune diseases are the consequence of complex interactions between a mosaic of host genetic factors and etiologic elements. Celiac disease (CD) is an autoimmune disease prevalent in 1% of the general population, but is unique on two accounts; a) the majority (90%) of individuals with CD have the HLA class II DQ2 allele, the others HLA-DQ8 and b) the etiologic agent is gluten proteins from wheat and related prolamins in barley and rye. The disease process is generally considered to be mediated by T cells that recognize HLA-DQ2 specific peptide sequences in gluten. There is currently no therapeutic treatment for CD. To this end, the inventors have identified a novel therapeutic target for CD and innate immune pathways in other inflammatory conditions.

### CROSS-REFERENCE TO RELATED APPLICATION

This application claims benefit of U.S. Provisional Application No. 61/060,783, filed Jun. 11, 2008, the disclosure of which is incorporated herein by reference.

### FIELD OF THE INVENTION

Complex interactions between host genetic factors and environmental elements are essential contributors to and/or influence the development of most, if not all autoimmune diseases. A genetic contribution to many of these diseases was identified a number of years ago with the observation that many individuals with different “autoimmune disorders” shared specific alleles of HLA class II genes. Advancements in identifying polymorphic genes across the human genome reveal additional genetic loci associated with specific autoimmune diseases, yet the strongest genetic component remains to be the HLA class II alleles. Still, there are several puzzling observations regarding the HLA class II allele association: a) while many of the diseases are organ- or tissue-specific, they share the same predisposing HLA class II alleles, b) although these alleles are common in Caucasian populations, only a minority of individuals develop disease and c) not all individuals with a specific disease have the allele most often associated with that disease. Extensive studies have attempted to define environmental factors and/or self antigens involved in the immune response that causes or influence destruction of specific tissues. There has been a long felt need for such definitions. In a number of diseases, antigens targeted by the adaptive immune response have been identified, and in some instances, the specific peptide sequence presented by the HLA class II alleles has been identified. However, there is little information regarding the function of genetic factors and etiologic agents in the activation of innate immune responses that trigger the cascade of events that are manifest in adaptive immunity.

In contrast to many autoimmune diseases, Celiac disease (CD) is unique at least because a) the majority (90%) of patients with this disease have the HLA class II DQ2 allele, the others HLA-DQ8 and b) there is a well established etiologic agent, glutens from wheat and related prolamins in barley and rye. Like other autoimmune diseases, it is not understood why the majority of individuals with the disease-associated alleles (HLA-DQ2, 35%) exposed to glutens in these dietary grains never develop CD. The disease process is generally considered to be mediated by T cells, and indeed T cell clones recognizing specific gluten-derived peptides (gliadins) presented by HLA-DQ2 and -DQ8 have been isolated from the gut of CD patients, demonstrating the adaptive immune response to the ingested proteins. Still, the potential role of these specific alleles and gliadins in the preceding innate immune response has not been addressed.

### BACKGROUND OF THE INVENTION

### Incidence and Pathology of Celiac Disease

Celiac disease (CD) is an autoimmune disorder triggered and influenced by glutens in the dietary grains wheat, barley and rye in genetically susceptible individuals (Sollid, Ann. Rev. Immunol., 18:53-81 (2000); Green et al., Lancet, 362(9381):383-391 (2003); and Sollid et al., Acta. Odontol. Scand., 59(3):183-186 (2001)). CD is a chronic inflammatory disorder of the small intestine triggered and influenced by dietary glutens in genetically susceptible individuals¹. It has an astounding prevalence in North American and European countries, affecting ˜1% of the general population and 8-15% of first-degree relatives and patients suffering from other autoimmune diseases such as type 1 diabetes (T1D), autoimmune thyroiditis, rheumatoid arthritis (RA), Sjogren's syndrome, Addison's disease and autoimmune hepatitis (Fasano et al., Arch. Intern. Med., 163(3):286-292 (2003); Betterle et al., Eur. J. Endocrinol., 154(2):275-279 (2006); Carroccio et al., Digestion, 60(1):86-88 (1999); and Pratesi et al., Scand. J. Gastroenterol., 38(7):747-750 (2003)). Given the increasing number of inflammatory disorders associated with gluten intolerance, CD provides a unique model for investigating the immunological events involved in its pathogenesis because it is the only disease for which the external (glutens) and genetic factors (specific HLA alleles) of disease susceptibility are clearly known.

CD is characterized by chronic inflammation of the small intestine resulting in atrophy of absorptive villi, hyperplasia of crypts, massive infiltration of intraepithelial lymphocytes and increased recruitment of lamina propria mononuclear cells, which often causes malabsorption and a plethora of clinical manifestations depending on the age of onset, extent of disease, and existence of additional tissue pathology. Classical symptoms include but are not limited to diarrhea, abdominal cramping, bloating and fatigue. CD is diagnosed upon detection of serum IgA antibodies to the autoantigen human tissue transglutaminase II (tTG), and confirmed by intestinal biopsy. The only treatment is lifelong adherence to a strict gluten-free diet, which presents a major challenge for individuals living with this disease as many struggle to follow total dietary compliance. Left untreated, severe problems such as vitamin deficiencies, osteoporosis and other extraintestinal complications may occur. Most affected individuals respond to the gluten-free diet with complete remission of tissue pathology, however some progress to refractory disease, increasing their risk of gastrointestinal malignancies (Brousse et al., Best Pract. Res. Clin. Gastroenterol., 19(3):401-412 (2005)).

Celiac disease results from convoluted interactions between multiple predisposing genes, dietary glutens, and innate and adaptive arms of the immune system, which may influence one another. Each of these factors pertinent to the pathogenesis of CD and related autoimmune diseases will be described briefly below.

In one aspect, the present invention provides a pepsin-trypsin digest of gliadin (PTG) which induces production of IL-23 by a subset of monocytes with highest levels in CD patients on a gluten-free diet, intermediate levels from HLA-DQ matched monocytes from normal individuals, and lowest levels from monocytes from HLA-DQ mismatched normal individuals, and the relative levels of other cytokines involved in the T_{H}17 response network follow this same pattern. In one aspect, IL-1β production is also induced by PTG and temporarily precedes IL-23 production, and this cytokine alone induces IL-23 production while the IL-1 receptor antagonist inhibits IL-23 production generated by monocytes exposed to PTG. This provides an non-limiting example of cytokine, pathway, genetic influence. In another aspect of the invention, the cytokine responses can only be generated with PTG and not with overlapping synthetic peptides from α-gliadin, but are also recapitulated with β-glucans from barley.

In one aspect of the invention, the IL-23/T_{H}17 innate immune response axis is activated by exposing defined populations of antigen presenting cells (APC) to gliadin proteins, and that this innate immune response influences the pathogenesis of CD in genetically predisposed individuals.

The present invention in an additional aspect establishes that the subset of monocytes and/or immature DC that produce IL-23 and related T_{H}17 cytokines in response to PTG exposure.

In one aspect, the present invention identifies, enumerates and compares the relative distribution of population(s) of monocytes, T cells, and their respective subsets associated with the IL 23/T_{H}17 pathway in peripheral blood mononuclear cells (PBMC) from patients with active and treated CD and HLA-DQ matched and HLA-DQ mismatched healthy individuals.

In one aspect, the present invention determines if the array and magnitude of cytokine and chemokine responses to PTG and other agents that activate the IL-23/T_{H}17 innate immune response is different in PBMC obtained from patients with active and treated CD and HLA-DQ matched and HLA-DQ mismatched healthy individuals.

### Genetics of Celiac Disease and Related Autoimmune Disorders

A remarkably strong association exists between susceptibility to CD and the specific HLA class II alleles HLA-DQ2 and HLA-DQ8, with 95% of patients expressing HLA-DQ2 (Sollid, Annu. Rev. Immunol., 18:53-81 (2000); and Margaritte-Jeannin et al., Tissue Antigens, 63(6):562-567 (2004). Moreover, individuals homozygous for the DQB*02 genes (DQ2/DQ2 and DQ2/DR7) are at increased risk for disease than those expressing the DQ8 (DR4)/X genotype (Louka et al., Hum. Immunol., 64(3):350-358 (2003); and Vader et al., Proc. Natl. Acad. Sci., U.S.A., 100(21):12390-12395 (2003)). These genes, located within the major histocompatibility complex on chromosome 6p21, encode cell-surface antigen-presenting proteins vital to the T cell-mediated process of adaptive immunity. Detection of gliadin-specific HLA-DQ2 and -DQ8-restricted CD4⁺ T cells clones in the active celiac lesion has helped explain the genetic prerequisite and inspired a decade of research establishing a central role for the adaptive immune response in CD (Sollid, Annu. Rev. Immunol., 18:53-81 (2000); Lundin et al., J. Exp. Med., 178(1):187-196 (1993); Lundin et al., Hum. Immunol., 41(4):285-291 (1994); and Molberg et al., Scand. J. Immunol., 46(3):103-109 (1997)). While these alleles are required for CD, they are not sufficient for pathogenesis given that the majority of individuals expressing HLA-DQ2/8 encounter gluten everyday and never develop disease.

Inheritance of the HLA-DQ alleles contributes 40% of the genetic requirement in CD, and the remaining 60% results from a complex mosaic of undetermined non-HLA genes (Sollid et al., Clin. Gastroenterol. Hepatol., 3(9):843-851 (2005)). Genetic association studies have identified numerous genes as candidates for susceptibility to CD, but confounding factors such as genetic heterogeneity, linkage disequilibrium, population stratification and limited sample size have generated conflicting results (Latiano et al., J. Pediatr. Gastroenterol. Nutr., 45(2):180-186 (2007); and Capilla et al., Tissue Antigens, 70(4):324-329 (2007)). These include the TNF-308A variant within the HLA complex on chromosome 6p21 (Sumnik et al., Diabetes Care, 29(4):858-863 (2006)), the CD28/CTLA4/ICOS cluster (CELIAC3) on chromosome 2q33 (Amundsen et al., Tissue Antigens, 64(5):593-599 (2004)), FcgRIIa*519GG on chromosome 1q23 (Alizadeh et al., Hum. Mol. Genet., 16(21):2552-2559 (2007)), as well as chromosomal loci 5q32-34 (CELIAC2) that encode IL17B and other cytokines (Ryan et al., Tissue Antigens, 65(2):150-155 (2005)) 3p21 that encodes the autoantigen GM1 ganglioside and the fractalkine receptor CX3CR1 (Neuhausen et al., Am. J. Med. Genet., 111(1):1-9 (2002)), 19p13 (CELIAC4) (Curley et al., Eur. J. Hum. Genet., 14(11):1215-1222 (2006); and Van Belzen et al., Gastroenterology, 125(4):1032-1041 (2003)), 11p11, 18q23, 7q31, 16q23, and 10q26 (King et al., Ann. Hum. Genet., 65(Pt. 4):377-386 (2001); and King et al., Ann. Hum. Genet., 64(Pt. 6):479-490 (2000)). Interestingly, the HLA class II alleles and several of the above mentioned loci have been implicated in other autoimmune diseases including T1D, autoimmune thyroiditis, Crohn's disease, RA, psoriasis and multiple sclerosis (MS), indicating a common disease process in the generation of adaptive immune responses that characterize these seemingly unrelated conditions. Additional genes include TNFR1, located on chromosome 12p13, a locus recently identified with CD in Bedouin kindred (Eller et al., Hum. Immunol., 67(11):940-950 (2006)); TNFR2, IL23R and NOD2 residing on chromosomes 1p36, 1p31 and 16q12 respectively, which have been implicated in Crohn's disease (Waschke et al., Am. J. Gastroenterol., 100(5):1126-1133 (2005); Pierik et al., Aliment Pharmacol. Ther., 20(3):303-310 (2004); Weersma et al., “ATG16L1 and IL23R Are Associated With Inflammatory Bowel Diseases but Not With Celiac Disease in The Netherlands”, Am. J. Gastroenterol. (2007); and Eckmann et al., Immunity, 22(6):661-667 (2005)); genes encoding the chemokine receptor CCR6 on chromosome 6q27, a locus associated with T1D, RA and MS, and its only known ligand CCL20 on chromosome 2q35-2q36 that has been linked to inflammatory bowel disease (IBD) (Nelson et al., Genomics, 73(1):28-37 (2001); and Rodriguez-Bores et al., World J. Gastroenterol., 13(42):5560-5570 (2007)); the IL-1 gene cluster on 2q previously reported in CD (Sumnik et al., Diabetes Care, 29(4):858-863 (2006); Moreno et al., Immunogenetics, 57(8):618-620 (2005); and Chowers et al., Clin. Exp. Immunol., 107(1):141-147 (1997)). A number of these genes encode products that are known to participate in inflammatory processes that are related to innate immune responses. Heretofore, the obligatory role of specific cytokines, chemokines and their cognate receptors in the IL23-mediated T_{H}17 pathway were not clearly functionally correlated with disease associated genes.

### Etiologic Agents of Celiac Disease

The external trigger for CD is enteric exposure to specific proteins in dietary grains known as prolamins, specifically wheat gliadins, barley hordeins, and rye secalins, which influence CD. This is illustrated by both the clinical and mucosal recovery of CD patients following a gluten-free diet (Fasano et al., Gastroenterology, 120(3):636-651 (2001)). Several features of these prolamins contribute to their immunogenicity. Their high proline content (>15%) confers resistance to proteolytic degradation in the gastrointestinal tract, and their abundant glutamine (>30%) residues provide copious targets for deamidation by the autoantigen tTG, which enhances the binding affinity to HLA-DQ2 at positions P4, P6 or P7 and P1, P4 or P9 for HLA-DQ8 (Tollefsen et al., J. Clin. Invest., 116(8):2226-2236 (2006); Johansen et al., Clin. Immunol. Immunopathol., 79(3):288-293 (1996); Vartdal et al., Eur. J. Immunol., 26(11):2764-2772 (1996); Johansen et al., Int. Immunol., 8(2):177-182 (1996); Godkin et al., Int. Immunol., 9(6):905-911 (1997); Kwok et al., J. Exp. Med., 183(3):1253-1258 (1996); and Kwok et al., J. Immunol., 156(6):2171-2177 (1996). In addition, these peptides naturally assume the left-handed polyproline II helical conformation preferred by all bound HLA-DQ2 and DQ8 ligands (Molberg et al., Eur. J. Immunol., 31(5):1317-1323 (2001); Shan et al., Science, 297(5590):2275-2279 (2002); and Kim et al., Proc. Natl. Acad. Sci., U.S.A., 101(12):4175-4179 (2004)). While these characteristics are important in generating the adaptive immune response that is manifest as CD, the structural or biochemical properties that lead to intestinal permeability and a role for innate cell activation in this process remain unclear.

The heterogeneous nature of wheat gliadin, which contains over 50 different proteins categorized as ω5-, ω1,2-α/β- or γ-gliadins based on amino acid sequence, composition and molecular weight (Wieser, Food Microbiol., 24(2):115-119 (2007)), has hindered both the identification of T cell epitopes that are targets of the adaptive immune response and properties required for innate cell activation (Turner et al., Protein Pept. Lett., 9(1):23-29 (2002)). Although lectin-like carbohydrate epitopes have not been detected in gliadin, higher molecular weight fractions (i.e., ω-gliadins) were recently shown to contain carbohydrates with the capacity to bind GM1 ganglioside, another autoantigen reported in CD (Alaedini et al., J. Neuroimmunol., 177(1-2):167-172 (2006)). GM1 is the ubiquitously expressed receptor for cholera toxin B (CTB) recognized for its potent immunomodulatory properties that are profoundly influenced by HLA class II haplotype in mouse models Nashar et al., Immunology, 106(1):60-70 (2002); Hirst et al., Symp. Ser. Soc. Appl. Microbiol., 27:26S-34S (1998); and Nashar et al., Vaccine, 13(9):803-810 (1995)). Indeed, Drago and colleagues have described a pronounced increase in the magnitude and duration of zonulin-mediated intestinal permeability in response to gliadin exposure in individuals with CD compared to that of healthy subjects of undetermined HLA status (Drago et al., Scand. J. Gastroenterol., 41(4):408-419 (2006)). Therefore, CD may ultimately result from an augmented innate immune response occurring in HLA-DQ2/DQ8 positive individuals upon ligation of glycosylated gliadins with GM1 on intestinal epithelial cells or intestinal dendritic cells (DC), similar to that generated by CTB/GM1 in H-2^{b} mice.

Clearly, environmental factors involved in development of CD are multifaceted and remain unclear. Several aspects of gluten exposure may influence the risk of CD occurrence, such as the amount of ingested gluten, the quality of ingested gluten, and the time at which gluten is included in infant feeding (Sollid, Nat. Rev. Immunol., 2(9):647-655 (2002)).

### Immunobiology of Celiac Disease and Related Autoimmune Diseases

Innate and Adaptive Immunity in CD

While the major genetic and environmental requirements for CD are known, the immunological events responsible for the deranged immune response are not well understood. Hallmarks of active disease include infiltration of cytotoxic intraepithelial lymphocytes (IEL) in the epithelium and gliadin-specific IFNγ-secreting CD4⁺ T cell clones restricted by HLA-DQ2 and DQ8 in the lamina propria (Lundin et al., J. Exp. Med., 178(1):187-196 (1993); Molberg et al., Scand. J. Immunol., 46(3):103-109 (1997); Halstensen et al., Scand. J. Immunol., 30(6):665-672 (1989); Maiuri et al., Am. J. Gastroenterol., 96(1):150-156 (2001); Meresse et al., Immunity, 21(3):357-366 (2004); and Meresse et al., J. Exp. Med., 203(5):1343-1355 (2006)). IEL differ from their lamina propria counterparts in both phenotype and function, and are considered more innate because they do not require TCR-specificity for activation. In healthy individuals, the majority of IEL are αβ T cells, 13% are γδT cells, 10% are CD8αα T cells, and 10% appear to be immature CD7⁺CD3⁻ T cells. This distribution is dramatically skewed in CD patients irrespective of diet with a permanent increase of γδ T cells and diminished proportion of CD7⁺CD3⁻ T cells (Eiras et al., Cytometry, 34(2):95-102 (1998); and Camarero et al., Acta Paediatr., 89(3):285-290 (2000)). Subsets of IEL are thought to be directly involved in the immediate destruction of IEC following consumption of dietary gluten in individuals with CD. The nonimmunodominant p31-43 epitope of α-gliadin has been demonstrated to activate both IL-15 dependent NKG2D-mediated killing of MICA/B⁺ IEC and IFNγ driven NKG2C-mediated killing of HLA-E⁺ IEC (Maiuri et al., Am. J. Gastroenterol., 96(1):150-156 (2001); and Meresse et al., J. Exp. Med., 203(5):1343-1355 (2006)). This same toxic epitope has also been reported to activate mucosal antigen presenting cells (APC), defined as CD3⁻COX2⁺CD86⁺ cells, isolated from intestinal biopsies of CD patients, but the mechanisms or cell population(s) involved were not determined (Maiuri et al., Am. J. Gastroenterol., 96(1):150-156 (2001)). While the adaptive immune response to gluten (and specifically immunodominant epitopes of the derivative protein, gliadin) has been well characterized in CD, the early events responsible for activation of the innate immune response have not been established.

Gliadin is thought to access the intestinal submucosa through the induction of zonulin signaling, which reversibly disrupts the integrity of epithelial tight junctions (Drago et al., Scand. J. Gastroenterol., 41(4):408-419 (2006)). Once the intestinal barrier is compromised, gliadin protein(s) or the peptides produced by enzymatic degradation of the protein must be acquired and processed by antigen presenting cells for presentation to gliadin-specific CD4⁺ T cells to initiate the adaptive immune response that characterizes CD (Weenink et al., Immunol. Cell Biol., 75(1):69-81 (1997)). The dynamic communication between IEC and intestinal dendritic cells (DC) are thought to regulate the processes of oral tolerance to harmless food antigens and commensal bacteria and immunity to harmful pathogens (FIG. 1). The aberrant response to gluten in CD immediately calls into question the maturation and activation state of mucosal DC subsets in these individuals. New evidence implicates a subset of lamina propria DQ2⁺ DC in the immunopathogenesis of CD. These DC were significantly increased in the lamina propria of untreated CD patients compared to treated CD patients and healthy donors, displayed an activated mature phenotype, and were exceptional at stimulating gliadin-specific T cell clones in vitro. Surface phenotyping suggest they derive from peripheral blood monocytes that continuously migrate through the intestinal mucosa (Raki et al., Gastroenterology, 131(2):428-438 (2006)). Indeed, Gliadin has also been reported to induce augmented levels of TNFα and IL-8 in blood monocytes from untreated CD patients compared to treated CD patients and healthy donors (Cinova et al., J. Clin. Immunol., 27(2):201-209 (2007)), and to mature monocyte-derived DC from healthy donors (Palova-Jelinkova et al., J. Immunol., 175(10):7038-7045 (2005)). These findings suggest that intolerance to gluten in CD may be initiated by accumulation and maturation of normally quiescent circulating monocytes upon encounter with gliadin, which can easily be manipulated in vitro to elucidate the mechanisms involved.

CD16⁺ Monocytes and Chronic Inflammation

Monocytes and their progeny are integral components of the innate immune system. In response to environmental antigens, conserved pattern recognition receptors (PRR) trigger cytokine production directing the immune response towards cellular and humoral adaptive immunity or immunologic tolerance to the encountered antigen (Williams et al., Leuk Lymphoma, 34(1-2):1-23 (1999)). Monocytes comprise two main subsets with distinct phenotypes and functions. In healthy individuals, 90-95% are classical monocytes, determined by CD14^{high}CCR2⁺CD16⁻GM1^{low}CX₃CR1^{low}, which serve as precursors for inflammatory DC recruited to sites of inflammation. The remaining 5-10%, characterized as CD14^{low}CCR2⁻CD16⁺GM1^{high}CX₃CR1^{high}, are considered resident monocytes because they constantly patrol non-inflated tissues in response to fractalkine (FKN/CX3CL1), a transmembrane chemokine constitutively expressed by IEC (Ziegler-Heitbrock, J. Leukoc. Biol., 81(3):584-592 (2007.); Yano et al., Acta. Med. Okayama, 61(2):89-98 (2007); and Moreno-Altamirano et al, Immunology, 120(4):536-543 (2007)). Moreover, this subset has been demonstrated to preferentially differentiate into migratory DC that mature upon exposure to yeast zymosan, serving as a primary first line of defense to invading pathogens (Randolph et al., J. Exp. Med., 196(4):517-527 (2002)). CD14^{low}CD16⁺ monocytes are also referred to as “proinflammatory” because they exhibit a more mature phenotype, have a greater capacity to produce the proinflammatory cytokines TNFα, IL-1 and IL-6, and are expanded in a number of inflammatory conditions including RA, Crohn's disease, Grave's disease, atherosclerosis, HIV (Ulrich et al., Am. J. Transplant., 8(1):103-110 (2008); Rahman et al., Crit. Care Med., 32(12):2457-2463 (2004); Abel et al., FEMS Microbiol. Immunol., 5(5-6):317-323 (1992); Belge et al., J. Immunol., 168(7):3536-3542 (2002); Ziegler-Heitbrock, Immunol. Today, 17(9):424-428 (1996); Abrahams et al., Arthritis Rheum, 43(3):608-616 (2000); Ancuta et al., J. Leukoc. Biol., 80(5):1156-1164 (2006); and Grip et al., Inflamm. Bowel Dis., 13(5):566-572 (2007)) and following excessive exercise (Steppich et al., Am. J. Physiol. Cell Physiol., 279(3):C578-C586 (2000)). Indeed, an increase of CD14⁺CD16⁺DQ2⁺ monocytes has been noted in the intestinal lesion of untreated CD patients; however their role in the immunopathogenesis of CD was neglected and warrants further exploration (Raki et al., Gastroenterology, 131(2):428-438 (2006)). Our novel findings indicate that this minor population of monocytes plays a fundamental role in the innate immune response to gliadin, and offer insight into the mechanisms involved.

IL-23/T_{H}17 Paradigm in Autoimmune Disease

In recent years, the emergence of the T_{H}17 hypothesis has replaced the T_{H}1 paradigm invoked to explain cell-mediated tissue damage in autoimmunity. A compelling association between the IL-23 mediated T_{H}17 pathway and tissue destruction in RA, psoriasis, Crohn's disease, ulcerative colitis and MS has not been described in CD (Fuss et al., Inflamm. Bowel Dis., 12(1):9-15 (2006); Kim et al., Scand. J. Rheumatol., 36(4):259-264 (2007); Kim et al., Rheumatology (Oxford), 46(1):57-64 (2007); Becker et al., J. Clin. Invest., 112(5):693-706 (2003); Lee et al., J. Exp. Med., 199(1):125-130 (2004); Piskin et al., J. Immunol., 176(3):1908-1915 (2006); and Vaknin-Dembinsky et al., J. Immunol., 176(12):7768-7774 (2006)).

IL-23 is a heterodimeric cytokine secreted by activated APC that consists of the IL-12/23p40 subunit and the protein IL-23p19 (Oppmann et al., Immunity, 13(5):715-725 (2000)); and Langrish et al., Immunol. Rev., 202:96-105 (2004)). It expands the memory subset of CD4⁺ T cells that secrete the tissue destructive cytokine IL-17, termed T_{H}17 (Steinman, Nat. Med., 13(2):139-145 (2007); and McKenzie et al, Trends. Immunol., 27(1):17-23 (2006)). This subset comprise a distinct lineage of CD4⁺ T cells generated in the presence of activated monocytes and IL-1 and IL-23 and in the absence of the T_{H}1 cytokine IFNγ, providing the necessary signals for STAT3-dependent transcription of the T_{H}17 master regulator RORC2 (Zhou et al., Nat. Immunol., 8(9):967-974 (2007); and Evans et al., Proc. Natl. Acad. Sci., U.S.A., 104(43):17034-17039 (2007)). T_{H}17 cells are characterized, for example, by surface expression of IL23R, CD45RO, CCR6 and CCR4 and their ability to produce IL-17A, IL-17F, IL-6, TNFα IL-21 and IL-22 (Steinman, Nat. Med., 13(2):139-145 (2007); Singh et al., J. Immunol., 180(1):214-21 (2008); Acosta-Rodriguez et al., Nat. Immunol., 8(6):639-646 (2007); and Wilson et al., Nat. Immunol., 8(9):950-957 (2007)). These exemplary cytokines enhance T cell priming and augment the proinflammatory mediators IL-1β, IL-6, TNFα, CXCL1 (GRO-1), CXCL2 (MIP-2α), and CXCL8 (IL-8), which recruit neutrophils and perpetuate inflammation (Schmidt-Weber et al., J. Allergy Clin. Immunol., 120(2):247-254 (2007)). Neutralization of IL-17, IL-1, IL-23 or CCL20 has been shown to ameliorate tissue pathology in autoimmune models of RA, Crohn's, and MS, illustrating the critical role of these cytokines/chemokines in the T_{H}17 response (Bush et al., Arthritis Rheum., 46(3):802-805 (2002); Hirota et al., J. Exp. Med., 204(12):2803-2812 (2007); Lubberts et al., Arthritis Rheum., 50(2):650-659 (2004); Lee et al., Gastroenterology, 133(1):108-123 (2007); Yen et al., J. Clin. Invest., 116(5):1310-1316 (2006); and Chen et al., J. Clin. Invest., 116(5):1317-1326 (2006)). In addition to activating T_{H}17 cells, IL-23 is thought to contribute to the chronicity of inflammation in an autocrine fashion, stimulating production of IL-1β, IL-6 and TNFα in IL-23R⁺ myeloid-derived APC (Hue et al., J. Exp. Med., 203(11):2473-2483 (2006); and Belladonna et al., J. Immunol., 168(11):5448-5454 (2002)).

Important for protection against microbial infection, studies have identified a number of agents capable of inducing IL-23 in monocytes and various sources of DC, depending on the cytokine milieu, the morphology of the microbe, and the differentiation state of the APC (Cooper, Eur. J. Immunol., 37(10):2680-2682 (2007)). These include cytokines, PGE₂, FasL and receptor/ligand pairs such as Dectin1/β-glucan, NOD2/peptidoglycan, and the combination of Dectin1 and TLR2 for yeast zymosan particles (Sheibanie et al., Faseb. J, 18(11):1318-1320 (2004); Liu et al., Rheumatology (Oxford), 46(8):1266-1273 (2007); LeibundGut-Landmann et al., Nat. Immunol., 8(6):630-638 (2007); Nakamura et al., FEMS Immunol. Med. Microbiol., 47(1):148-154 (2006); and van Beelen et al., Immunity, 27(4):660-669 (2007)). Despite the undisputed advantages of studying CD as a model of autoimmunity, the effect of gliadins on the IL-23/T_{H}17 is unknown.

### SUMMARY OF THE INVENTION

Autoimmune diseases are the consequence of complex interactions between a mosaic of host genetic factors and etiologic elements. Celiac disease (CD) is an autoimmune disease prevalent in 1% of the general population, but is unique on two accounts; a) the majority (90%) of individuals with CD have the HLA class II DQ2 allele, the others HLA-DQ8 and b) the etiologic agent is gluten proteins from wheat and related prolamins in barley and rye. The disease process is generally considered to be mediated by T cells that recognize HLA-DQ2 specific peptide sequences in gluten. CD14^{low}CD16⁺ monocytes in PBMC from patients and controls produce the cytokines and chemokine associated with this pathway (IL-23, IL-1β, IL-6, TNFα, MIP-3α) when exposed to pepsin-trypsin digest of gliadin (PTG). Cytokine levels are significantly higher in cells from treated CD patients than controls. While levels detected in HLA-DQ2 matched controls were reduced compared to patients, they were considerably higher than controls not having the disease associated alleles. Gliadin activation of the IL-23/T_{H}17 innate immune response pathway plays a fundamental role in the pathogenesis of CD. We confirmed the cell source(s) of the IL-23 response to PTG, classify, enumerate and compare the relative distribution of population(s) of monocytes, T cells, and their respective subsets associated with the IL-23/T_{H}17 pathway in PBMC from CD patients with active disease, treated disease, and HLA-DQ2 matched and HLA-DQ mismatched healthy individuals and establish the array and magnitude of cytokine/chemokine responses to PTG from the different patient groups. We identify phenotypic and functional properties of cell populations that respond to gliadins with induction of cytokines/chemokines comprising the IL-23/T_{H}17 innate immune response, and establish differences that distinguish patients with CD from healthy populations. We identify therapeutic targets for this disease and innate immune pathways in other inflammatory conditions.

Accordingly, it is an object of the invention to provide a cell population comprising at least one cell, wherein said cell influences celiac disease. An exemplary cell population responds to gliadins. Further, an exemplary cell population produces cytokines, such as T_{H}17 cytokines. A further exemplary object of the invention is a cell population producing cytokines wherein said cytokines influence IL-1β. Another exemplary object of the invention is a cell population producing cytokines wherein said cytokines influence IL-1β, and wherein said population influences IL-1RA. The present invention also provides a pepsin-trypsin digest of gliadin (PTG) which induces production of IL-23 by a subset of monocytes with highest levels in CD patients on a gluten-free diet, intermediate levels from HLA-DQ matched monocytes from normal individuals, and lowest levels from monocytes from HLA-DQ mismatched normal individuals, and the relative levels of other cytokines involved in the T_{H}17 response network follow this same pattern. In one object, IL-1β production is also induced by PTG and temporarily precedes IL-23 production, and this cytokine alone induces IL-23 production while the IL-1 receptor antagonist inhibits IL-23 production generated by monocytes exposed to PTG. There is provided a non-limiting example of cytokine, pathway, genetic influence. In another object of the invention, the cytokine responses can only be generated with PTG and not with overlapping synthetic peptides from α-gliadin, but are also recapitulated with β-glucans from barley. An additional exemplary cell population produces chemokines.

A second object of the invention is a method influencing immunity in an individual with celiac disease. An exemplary method includes influencing T_{H}17 cytokines. Further, an exemplary method includes influencing innate immunity. An additional object of the invention is a method influencing immunity in an individual with celiac disease wherein IL-1β is influenced. Yet another exemplary object includes a method influencing immunity in an individual with celiac disease wherein IL-1RA is influenced. In one object, the present invention identifies, enumerates and compares the relative distribution of population(s) of monocytes, T cells, and their respective subsets associated with the IL 23/T_{H}17 pathway in peripheral blood mononuclear cells (PBMC) from patients with active and treated CD and HLA-DQ matched and HLA-DQ mismatched healthy individuals. In one object, the present invention determines if the array and magnitude of cytokine and chemokine responses to PTG and other agents that activate the IL-23/T_{H}17 innate immune response is different in PBMC obtained from patients with active and treated CD and HLA-DQ matched and HLA-DQ mismatched healthy individuals.

A third object of the invention is a composition for improving immune function, comprising an immune modulating component, comprising a first agent, wherein said first agent modulates immunity. An exemplary composition includes a first agent that influences celiac disease in a patient wherein said patient has at least one cell that responds to gliadins. Further, a therapeutic composition is an object of the present invention and comprises a first agent, wherein said first agent influences celiac disease in a patient wherein said patient has at least one cell that responds to gliadins. An exemplary therapeutic composition includes a first agent that inhibits at least one cell that responds to gliadins. An exemplary therapeutic composition includes a first agent that inhibits cytokine production of at least one cell that responds to gliadins.

A fourth object of the invention is a diagnostic method, comprising contacting at least one cell wherein said cell is obtained from an individual with celiac disease, further comprising contacting at least one cell wherein said cell is obtained from an individual without celiac disease, and further comprising comparing said cell from an individual with celiac disease to said cell from an individual without celiac disease. An exemplary diagnostic method includes influencing T_{H}17 cytokines. An exemplary diagnostic method includes influencing innate immunity.

A fifth object of the invention is a composition for improving immune function. An exemplary object of the invention includes a composition for improving immune function that includes an immune modulating component. An exemplary object of the invention includes a composition for improving immune function that includes an immune modulating component comprising a first agent, wherein said first agent modulates immunity, wherein said immunity involves IL-1β. An additional exemplary object of the invention is a composition for improving immune function, comprising an immune modulating component, comprising a first agent, wherein said first agent modulates immunity, wherein said immunity involves IL-1RA.

A sixth object of the invention includes a diagnostic method, wherein said method includes contacting at least one cell obtained from an individual with celiac disease and contacting at least one cell wherein said second cell is obtained from an individual without celiac disease, and comparing the cell (or cells) from an individual with celiac disease to a cell (or cells) from an individual without celiac disease. An exemplary embodiment includes a diagnostic method wherein cellular IL-1β from an individual without celiac disease is compared to IL-1β from an individual with celiac disease. An exemplary object of the invention also includes a diagnostic method comprising contacting at least one cell wherein said cell is obtained from an individual with celiac disease, further comprising contacting at least one cell wherein said cell is obtained from an individual without celiac disease, further comprising comparing said cell from an individual with celiac disease to said cell from an individual without celiac disease, and further comprising comparing IL-1RA from an individual without celiac disease to IL-1RA from an individual with celiac disease.

A seventh object of the invention includes a method of reducing inflammation in a subject with celiac disease comprising administering to said subject an IL-1 antagonist or an IL-1 inhibitor wherein said administering of said IL-1 antagonist or said IL-1 inhibitor reduces inflammation in said subject with celiac disease. An exemplary object of the invention includes a method of reducing inflammation, wherein the inflammation is IL-23 mediated. An exemplary object of the invention includes a method of reducing inflammation, wherein an IL-1 antagonist or an IL-1 inhibitor is administered and elicits a reduction of IL-23 or IL-1β. Yet another exemplary object of the invention includes a method of reducing inflammation, wherein said method includes administering an IL-1 antagonist or an IL-1 inhibitor that causes a reduction of IL-23. A further object of the invention includes a method of reducing inflammation, wherein said method includes administering an IL-1 antagonist or an IL-1 inhibitor and wherein said IL-1 antagonist or said IL-1 inhibitor is IL-1ra.

The objects of the present invention as summarized and taught herein are exemplary and non-limiting.

### BRIEF DESCRIPTION OF THE DRAWINGS

FIG. 1 illustrates that subsets of mucosal DC regulate antigenic tolerance and immunity in the intestine. CX3CR1+ DC form tight junctions with IEC for continuous luminal sampling and populate the entire lamina propria and dome regions of Peyer's patches. CCR6+ DC reside in Peyer's patches and are recruited to the subepithelial dome region in response to pathogen-triggered CCL20 production. FAE means follicle associated epithelium.

FIGS. 2A-2E illustrate that monocytes are a major source of T_{H}17 cytokines triggered by exposure to PTG, however IFNγ treatment allows immDC to respond similarly. IL-23 data is shown as the mean of 5 independent experiments; IL-1β, TNFα, CCL20 and IL-6 are representative of 3 independent experiments. Exposure to β-glucan produced similar results.

FIGS. 3A-3D illustrate the obligatory role of IL-1 signaling in activation of IL-23 innate response in purified monocytes. Pretreatment with IL-1ra blocks PTG (FIG. 3A) and β-glucan (FIG. 3B) induced IL-23 production. Dose dependent IL-23 response to exogenous IL-1β alone in monocytes (FIG. 3C). IFN-γ treated DC response to PTG is independent of IL-1 signaling (FIG. 3D). FIG. 3B is one representative of four independent experiments. FIG. 3D represents one of three independent experiments.

FIGS. 4A-4E show that the CD14^{low}CD16⁺ subset of circulating monocytes is required for PTG-induced IL-1β and IL-23 production. Monocytes purified by elutriation were sorted into CD14^{low} and CD14^{high} subsets and cultured in the absence or presence of 100 ug/ml PTG for 24 h. Only the CD14^{low}CD16⁺ cells produced IL-1β and IL-23 upon exposure to PTG.

FIG. 5 illustrates CD14^{low}CD16⁺ cells increased in peripheral blood of CD patients in remission on GFD and decreased in CD patients with active disease.

FIGS. 6A-6E shows PBMC from healthy individuals produce IL-23 and additional cytokines/chemokines associated with induction of T_{H}17 cells upon exposure to PTG. Addition of IL-1ra inhibits PTG activation of the IL-23/T_{H}17 innate immune response, and β-glucan and IL-1β alone have the capacity to recapitulate this innate response. These data are representative of 6 independent experiments.

FIGS. 7A-7B illustrate kinetics and influence of HLA status on IL-23/T_{H}17 innate immune response pathway. After 24 h, PBMC from HLA-DQ2 positive healthy individuals produce substantially more IL-23 in response to PTG and β-glucan than HLA-DQ2 negative normal donors. Background levels were subtracted before plotting the effects of PTG or β-glucan.

FIGS. 8A-8C show gliadin-induced cytokine production is significantly higher in PBMC from CD patients than DQ2⁺ healthy individuals. Background levels from medium alone were subtracted from PTG before calculating the means.

FIG. 9 illustrates gliadin induces robust production of IL-23 and related proinflammatory cytokines in PBMC from CD patients. FIG. 9A shows PBMC from CD patients generate significantly higher amounts of IL-23, IL-1β and TNFα in response to PTG stimulation than HLA-DQ2⁺ healthy individuals. PTG substantially reduces secretion of the anti-inflammatory cytokine IL-1ra in CD patients but not in healthy individuals, and only stimulates significant levels of IL-6 in CD patients. PBMC from 7 CD patients and 6 HLA-DQ2⁺ healthy individuals (HD) were incubated with or without PTG (100 μg/ml) for 48 h, and cell-free culture supernatants analyzed for production of IL-1β, IL-1ra, IL-6, IL-12p70, IL-23 and TNFα. Together, these data illustrate that proinflammatory cytokine responses to PTG are augmented in HLA-DQ2⁺ individuals with CD compared to those without disease. Error bars indicate +s.d. FIG. 9B shows PTG stimulation of IL-23 and IL-1β production is dose dependent. PBMC from CD patients were cultured with or without 25, 100, 250 or 500 μg/ml PTG for 24 h. Increasing doses of β-glucan from barley served as a positive control. Concentrations of IL-23 and IL-1β were quantified by ELISA. Data represents mean values from 3 independent experiments. Error bars indicate +s.d.

FIG. 10 illustrates IL-1 cytokines regulate the IL-23 response in vitro. FIG. 10A shows that the addition of IL-1ra significantly inhibits IL-23 and IL-1β responses to PTG and the positive control, β-glucan. PBMC were incubated with or without 0.5 μg/ml IL-1ra for 1 h prior to stimulation with PTG or β-glucan for 20 h. Secretion of IL-23 and IL-1β were determined by ELISA. These data are mean values from 10 independent experiments. Error bars indicate +s.d. FIG. 10B shows IL-1β alone stimulates PBMC to produce IL-23, however its capacity to do so is much lower (˜10-fold) than that of PTG or β-glucan. PBMC were cultured in the absence or presence of 5 ng/ml IL-1β for 20 h, and supernatants tested by IL-23 ELISA. These results represent the mean of 10 independent experiments. Error bars indicate +s.d.

FIG. 11 illustrates that monocytes are the cell source of IL-23 and related proinflammatory mediators produced in response to in vitro gliadin stimulation. Highly purified lymphocytes, monocytes or monocyte-derived immDC were incubated with or without PTG (100 μg/ml) for 24 h, and supernatants analyzed for production of IL-1μ, IL-6 IL-23, TNFα and CCL20. PTG directly activates monocytes, and not lymphocytes or immature DC, to secrete IL-23. IL-1β, TNFα, IL-6 and CCL20 responses were also generated by monocytes exposed to PTG, and not their progeny DC. IL-23 data represent the mean of 5 independent experiments. IL-1β, TNFα and IL-6 data represent the means of 3 independent experiments. CCL20 data is one representative of 3 independent experiments. Error bars indicate +s.d.

FIG. 12 illustrates the IL-1 system regulates IL-23 production in human monocytes. FIG. 12A shows IL-1ra significantly inhibited IL-23 responses from monocytes exposed to PTG and the positive control, β-glucan. Highly purified monocytes were incubated with or without 0.5 μg/ml IL-1ra prior to addition of PTG or β-glucan for 20 h. These results represent the means of 5 independent experiments. Error bars indicate +s.d. FIG. 12B shows that IL-1β alone directly activates monocytes to secrete IL-23 in a dose dependent manner, however its capacity to do so is greatly reduced (˜10-fold) compared to that of PTG or β-glucan. Purified monocytes were treated with and without 0.5, 5 or 50 ng/ml rhIL-1β for 20 h, and culture supernatants were analyzed for IL-23 production. These results represent the means of 5 independent experiments. P values compare IL-1β data sets to medium alone. Error bars indicate +s.d.

### DETAILED DESCRIPTION OF THE INVENTION

### Definitions

Unless otherwise noted, technical terms are used according to conventional usage. Definitions of common scientific technical terms may be found, for example, in McGraw-hill Dictionary of Scientific & Technical Terms published by McGraw-hill Healthcare Management Group; Benjamin Lewin, Genes VIII, published by Oxford University Press; Kendrew et al. (eds.), The Encyclopedia of Molecular Biology, published by Blackwell Publishers; and Robert A. Meyers (ed.), Molecular Biology and Biotechnology: a Comprehensive Desk Reference, published by Wiley, John & Sons, Inc; and other similar technical references.

As used herein, “a” or “an” may mean one or more. As used herein in the claim(s), when used in conjunction with the word “comprising”, the words “a” or “an” may mean one or more than one. As used herein “another” may mean at least a second or more. Furthermore, unless otherwise required by context, singular terms shall include pluralities and plural terms shall include the singular.

As used herein, the term “agent” is a molecular entity including, for example, a small molecule (especially small organic molecules that satisfy the constraints of Lipinski's Rules (Lipinski, C. A. et al. (1997) “Experimental And Computational Approaches To Estimate Solubility And Permeability In Drug Discovery And Development Settings,” Adv. Drug Del. Rev, 23:3-25; Lipinski, C. A. et al. (2001) “Experimental And Computational Approaches To Estimate Solubility And Permeability In Drug Discovery And Development Settings,” Adv. Drug Del. Rev. 46,3-26; Oprea, T. I. et al. (2001) “Is There A Difference Between Leads And Drugs? A Historical Perspective,” J. Chem. Inf. Comput. Sci. 41:1308-1315; Arup, K. et al. (1999) “A Knowledge-Based Approach in Designing Combinatorial or Medicinal Chemistry Libraries for Drug Discovery,” J. Combin. Chem. 1:55-68), a nucleic acid (e.g., an oligonucleotide, and in particular, a siRNA, a shRNA an expression cassette, an antisense DNA, an antisense RNA, etc.), protein, peptide, antibody, antisense drug, or other biomolecule that is naturally made, synthetically made, or semi-synthetically made and is used alone or in combination with other therapies or methods for the treatment of a pathological condition of the invention (including, for example, a CD).

As used herein, an “IL-1 antagonist,” “IL-1 inhibitor,” or an obvious derivation thereof is an agent that is capable of reducing the effective amount of endogenous biologically active IL-1β, TNFα, or IL-23 by, for example, reducing the amount of IL-1β, or by inhibiting the binding of IL-1β to its receptor. In particular aspects, an IL-1 antagonist or IL-1 inhibitor is capable of reducing the effective amount of endogenous biologically active IL-23.

As used herein, “treat” and all its forms and tenses (including, for example, treat, treating, treated, and treatment) refers administering an agent of the invention to a subject in need thereof for both therapeutic treatment or prophylactic or preventative treatment. A subject in need of treatment includes those already with a pathological condition of the invention (including, for example, a CD) as well as those in which a pathological condition of the invention is to be prevented.

### DESCRIPTION OF INVENTION

We are the first to correlate IL-23 mediated inflammation in the pathogenesis of CD. More than a decade of research has defined the T cell response to gluten-derived gliadin peptides in CD, yet the early events that initiate its activation are not well understood (Lundin et al., Hum. Immunol., 41:285-91 (1994); Tollefsen et al., J. Clin. Invest., 116:2226-2236 (2006); and van de Wal et al., Proc. Natl. Acad. Sci., U.S.A., 95:10050-10054 (1998)). The compromised intestinal epithelial barrier that characterizes CD allows gliadin access to the intestinal submucosa, where it must be acquired and processed by antigen presenting cells (APC) for presentation and activation of gliadin-specific CD4⁺ T cells. While augmented levels of zonulin and potent inflammatory cytokines IL-1β and TNFα have all been reported to increase intestinal permeability by disrupting the integrity of tight junctions in individuals with CD and other forms of inflammatory bowel disease (IBD), the precise mechanisms involved remain to be determined (Drago et al., Scand. J. Gastroenterol., 41:408-419 (2006); Al-Sadi et al., J. Immunol., 178:4641-4649 (2007); and Ma et al., Am. J. Physiol. Gastrointest. Liver Physiol., 286:G367-G376 (2004)).

A dynamic relationship between intestinal epithelial cells (IEC) and dendritic cells (iDC) regulates the processes of immunologic tolerance to harmless food and commensal antigens and adaptive immunity to pathogens (Kelsall et al., Immunol. Rev., 206:132-148 (2005)). The aberrant response to dietary glutens in CD immediately calls into question the maturation and activation state of iDC in these individuals. Indeed, a subset of activated lamina propria DQ2⁺ DC derived from circulating blood monocytes was recently implicated in the pathogenesis of CD (Raki et al., Gastroenterology, 131:428-438 (2006)). Moreover, circulating monocytes from CD patients have been demonstrated to produce substantially more TNFα and IL-8 in response to gliadin than monocytes from healthy individuals (Cinova et al., J. Clin. Immunol., 27:201-209 (2007)). Together, these findings suggest that CD ultimately results from accumulation of normally quiescent circulating monocytes that are activated upon encounter with gliadin in the gut.

Monocytes and their progeny are integral components of the innate immune system. In response to environmental antigens, conserved pattern recognition receptors (PRR) trigger cytokine production directing the immune response to the encountered antigen (Williams et al., Leuk. Lymphoma., 34:1-23 (1999)). New evidence shows that activated monocytes producing IL-1β and IL-23 are the most potent stimulators of the memory subset of pathogenic T helper cells (termed Th17) that secrete tissue destructive cytokines IL-17, IL-21 and IL-22 (Acosta-Rodriguez et al., Nat. Immunol., 8:942-949 (2007); Steinman, Nat. Med., 13:139-145 (2007); and McKenzie et al., Trends Immunol., 27:17-23 (2006)).

IL-1 was one of the first cytokines to be described and has since proved to be an important mediator of multiple immunologic processes throughout the body, including inflammatory conditions in the gut (Ligumsky et al., Gut, 31:686-689 (1990)). The IL-1 family consists of proinflammatory cytokines IL-1α and IL-1β and anti-inflammatory IL-1ra, which prevents IL-1 signaling by binding the active IL-1 receptor (IL-1RI) (Schreuder et al., Nature, 386:194-200 (1997)). An imbalance between IL-1β and IL-1ra, resulting from amplified levels of IL-1β has been associated with inflammation in CD (Fornari et al., Am. J. Gastroenterol., 93:413-418 (1998)). Interestingly, elimination of dietary glutens significantly increases levels of IL-1ra in these individuals without substantially altering IL-1β, suggesting that individuals with CD inherently produce more IL-1β and IL-1ra, and that dietary glutens may induce inflammation by shifting the balance toward IL-1β in individuals with CD.

IL-23 is a relatively new inflammatory cytokine composed of the IL-12/23p40 subunit and the IL-23p19 protein that is preferentially secreted in specific tissues by APC (Oppmann et al., Immunity, 13:715-725 (2000); and Uhlig et al., Immunity, 25:309-318 (2006)). It perpetuates chronic inflammation by stimulating both adaptive and innate cells to produce additional proinflammatory mediators (Hue et al., J. Exp. Med., 203:2473-2483 (2006)). CD has been considered a typical Th1 disease, however emergence of the IL23-Th17 paradigm has prompted reanalysis of cell-mediated tissue damage previously attributed to the IL12-Th1 axis, and emphasized the decisive role of the innate arm in adaptive immunity. Although novel studies have detected augmented levels of IL-23 in rheumatoid arthritis, psoriasis. Crohn's disease, ulcerative colitis and multiple sclerosis, and other cytokines associated with Th17-mediated inflammation (IL-1β, IL-6, IL-15 and TNFα) have been implicated in the pathogenesis of CD, an association with IL-23 has not yet been reported (Fornari et al., Am. J. Gastroenterol., 93:413-418 (1998); Kim et al., Scand. J. Rheumatol., 36:259-264 (2007); Becker et al., J. Clin. Invest., 112:693-706 (2003); Piskin et al, J. Immunol., 176:1908-1915 (2006); Cua et al., Nature, 421:744-748 (2003); J. Invest. Dermatol., 127:2495-2497 (2007); Yen et al., J. Clin. Invest., 116:1310-1316 (2006); Chowers et al., Clin. Exp. Immunol., 107:141-147 (1997); and Thomas et al., J. Immunol., 176:2512-2521 (2006)).

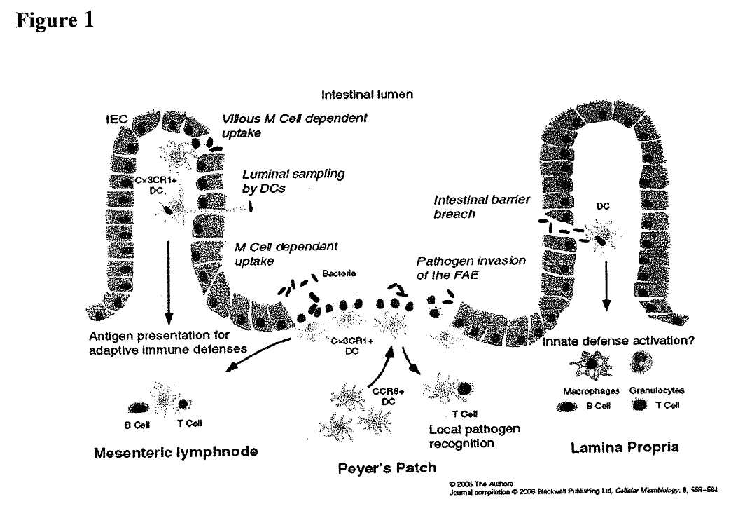

Given the strong genetic requirement associated with CD, we investigated gliadin's capacity to activate the IL-23 pathway in HLA-DQ2⁺ individuals with and without CD. We predicted that gliadin would induce increased levels of IL-23 and related inflammatory cytokines in HLA-DQ2⁺ individuals with CD compared to healthy individuals. To test this hypothesis, we exposed PBMC from CD patients and HLA-DQ2⁺ healthy individuals to a pepsin-trypsin digest of gliadin (PTG) and analyzed culture supernatants for IL-1β, IL-1ra, IL-6, IL-12p70, IL-23 and TNFα. We discovered that PTG stimulated production of IL-23, IL-1β, IL-6 and TNFα and reduced secretion of IL-1ra in all donors tested, however levels of IL-1β, IL-23, IL-6 and TNFα were significantly higher, and IL-1ra substantially reduced, in CD patients (FIG. 1A). Importantly, PTG did not induce IL-12p70 in any of the donors tested (negative data not shown). These results confirm that gliadin stimulates robust production of IL-1β and TNFα in individuals with CD (Cinova et al., J. Clin. Immunol., 27:201-209 (2007); and Fornari et al., Am. J. Gastroenterol., 93:413-418 (1998)) and demonstrate gliadin's ability to disrupt the balance between IL-1β and IL-1ra by simultaneously inducing high levels of IL-1β and decreased levels of IL-1ra. Moreover, our novel findings strongly advocate a role for IL-23 mediated inflammation in the pathogenesis of CD.

In order to demonstrate that production of these potent mediators depended on gliadin exposure, dose response curves were generated with PTG or β-glucan, an agent known to activate the IL-23 pathway. Both stimuli induced dose-dependent production of IL-1β and IL-23, although PTG proved to be far more effective as evidenced by detectable levels of IL-23 achieved with 100 μg/ml versus 500 μg/ml of β-glucan (FIG. 1B). These stimulatory effects of PTG were not due to endotoxin contamination, since the presence of LPS in this preparation of PTG was ruled out in earlier studies (Thomas et al., J. Immunol., 176:2512-2521 (2006)).

Several immunodominant epitopes of α-gliadin that preferentially bind HLA-DQ2 and DQ8 molecules as well as an innate peptide p31-43 have been implicated in the pathogenesis of CD (Maiuri et al., Lancet, 362:30-37 (2003)). To determine if any of these epitopes were involved in activation of the innate immune response, we incubated PBMC with synthetic overlapping peptides spanning the entire sequence of α-gliadin. None of the overlapping peptides tested individually or in combination stimulated secretion of IL-1β or IL-23, indicating that other subtypes of gliadin (γ- or ω-gliadin) or additional properties of gliadin are required for induction of these cytokines (negative data not shown). Since gliadin is a glycoprotein and β-glucan recapitulates the inflammatory cytokine response generated by PTG, posttranslational modifications are likely necessary for pattern recognition and activation of APC.

### Methods of Treatment

In particular aspects of the invention, methods of treatment are drawn to inhibiting the innate immune response that is responsible for causing cell injury or dysfunction in autoimmunity. In further particular aspects, inhibiting the innate immune response is inhibiting IL-23 mediated inflammation in autoimmunity. IL-23 mediated inflammation in autoimmunity can be the cause of cell injury or dysfunction in autoimmunity in a number of diseases, including, for example, rheumatoid arthritis, Crohn's disease, lupus, Hashimoto's thyroiditis, Sjogren's syndrome, multiple sclerosis, Graves' disease, Guillain-barre, ulcerative colitis, psoriasis, and CD. In certain aspects of the invention, IL-23 mediated inflammation in autoimmunity is a cause of cell injury or dysfunction in CD. As described herein and throughout the specification, the inventors are the first to describe IL-23 mediated inflammation in the pathogenesis of CD. The inventors are also the first to demonstrate that the IL-1 system regulates IL-23, and illustrate the powerful anti-inflammatory effects of IL-1ra on induction of IL-23. These two novel findings support novel methods of treating CD. Such methods include, for example, a method comprising administering an IL-1 antagonist or IL-1 inhibitor to a subject in need thereof.

An IL-1 antagonist or IL-1 inhibitor of the invention include, for example, a receptor-binding peptide fragment of IL-1, an IL-1, IL-1β or IL-1 receptor antibody, an IL-1ra polypeptide, an IL-1β converting enzyme (ICE) inhibitor (US Patent Application Publication No. 20090022733); IL-1ra (KINERET); sIL-1ra, icIL-1raI, icIL-1raII, and other IL-1 receptor antagonists described in U.S. Pat. No. 5,739,282; rilonacept (U.S. Pat. No. 6,927,044 and US Patent Application Publication No. 20090123446); IL-1β binding antibody or IL-1β binding fragment that bind selectively to IL-1β (U.S. Pat. No. 7,491,392 and US Patent Application Publication No. 20090060923); a human IL-1 receptor type 1 antibody (U.S. Pat. No. 7,438,910); a noncompetitive antagonist of IL-1 receptor, including RYTVELA (SEQ ID NO:1), MKLPVHKLY (SEQ ID NO:2), VGSPKNAVPPV (SEQ ID NO:3), AND WTLDGKKPDDL (SEQ ID NO:4) (Quiniou et al. J Immunol. 2008 May 15;180(10):6977-87).

In certain aspects of the invention an IL-1ra polypeptide includes a form of IL-1ra described in U.S. Pat. No. 5,075,222 and modified forms and variants including those described U.S. Pat. No. 5,922,573, and PCT Patent Application Publication Nos. WO 91/17184, WO 92 16221, and WO 96 09323; IL-1β converting enzyme (ICE) inhibitors include peptidyl and small molecule ICE inhibitors including those described in PCT Patent Application Publication Nos. WO 91/15577, WO 93/05071, WO 93/09135, WO 93/14777 and WO 93/16710, and European patent application 0 547 699; non-peptidyl compounds include those described in PCT Patent Application Publication No. WO 95/26958, U.S. Pat. Nos. 5,552,400, 6,121,266, and Dolle et al., J. Med. Chem., 39, pp. 2438-2440 (1996); and additional ICE inhibitors are described in U.S. Pat. Nos. 7,417,029, 6,162,790, 6,204,261, 6,136,787, 6,103,711, 6,025,147, 6,008,217, 5,973,111, 5,874,424, 5,847,135, 5,843,904, 5,756,466, 5,656,627, 5,716,929. Said references are incorporated by reference herein in their entireties.

### Cell Source of IL-23/T_{H}17 Innate Response to PTG

PTG Directly Stimulates the Production of T_{H}17-Related Cytokines in Monocytes

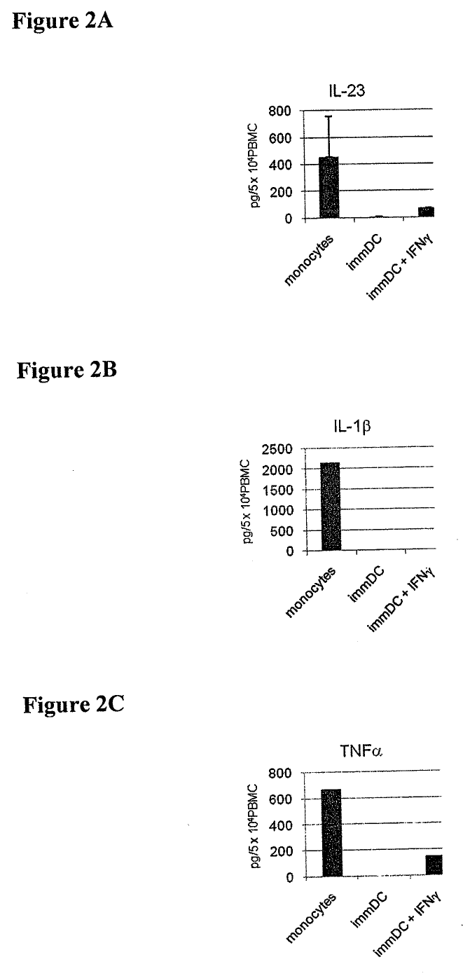

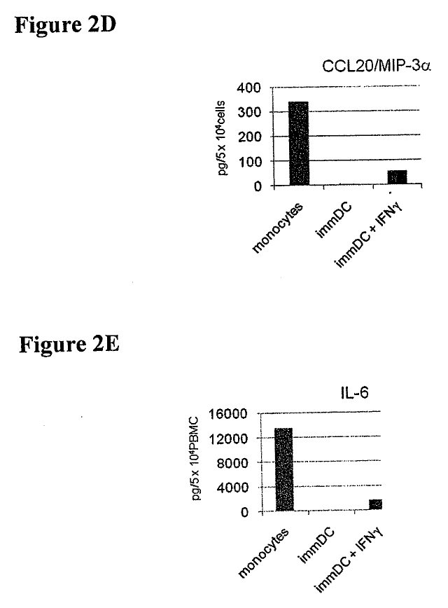

IL-23 is only secreted by activated APC such as monocytes, macrophages and DC, which reduces the number of possible cell sources considerably. To determine which of these cell populations was the source of IL-23 in response to PTG, purified lymphocytes, monocytes, or monocyte-derived DC cultured with GM-CSF and IL-4, were incubated in the presence or absence of PTG for 24 h, at which time supernatants were collected for IL-23, IL-1β, IL-6, IFNγ and TNFα analysis. Under these conditions, both PTG- and β-glucan-induced secretion of IL-23, IL-1β, CCL20, TNFα and IL-6 was confined to monocytes, identifying a readily available target population for further investigation, and indicating a direct interaction of PTG with its PRR(s) whose expression must be limited to this population (FIGS. 2A-2E). Since neither PTG nor β-glucan activated cytokine production in DC differed from monocytes, we examined the response after pretreatment with IFNγ, given that this treatment has been necessary for the IL-23 response to a variety of agents including β-glucan. immDC were cultured in the presence or absence of IFNγ for 18 h prior to addition of PTG, after which supernatants were subjected to cytokine analysis as described above. Similar to monocytes, IFNγ-treated immDC produced IL-23, IL-6, TNFα and CCL20 in response to PTG, implying that IFN-γ upregulates the PRR(s) or cytoplasmic signaling components required for the response in immDC, and calls into question the properties of PTG and its cognate PRR expressed on monocytes and IFNγ-treated immDC mediating this response. In contrast to monocytes, the magnitude of the immDC response was notably reduced and lacked IL-1β altogether, suggesting that PTG activates distinct signaling cascades in the different cell types that ultimately determine the fate of the adaptive immune response to PTG (FIGS. 2A-2E). Moreover, these findings provide insight to the mechanisms in which IFNγ counteracts IL-1 dependent T_{H}17 responses.

IL-1β Regulates the IL-23/T_{H}17 Innate Immune Response Triggered by PTG in Monocytes

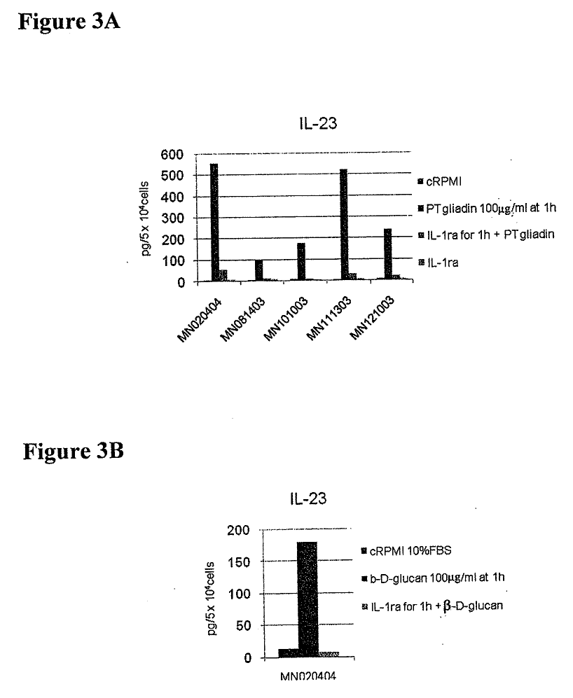

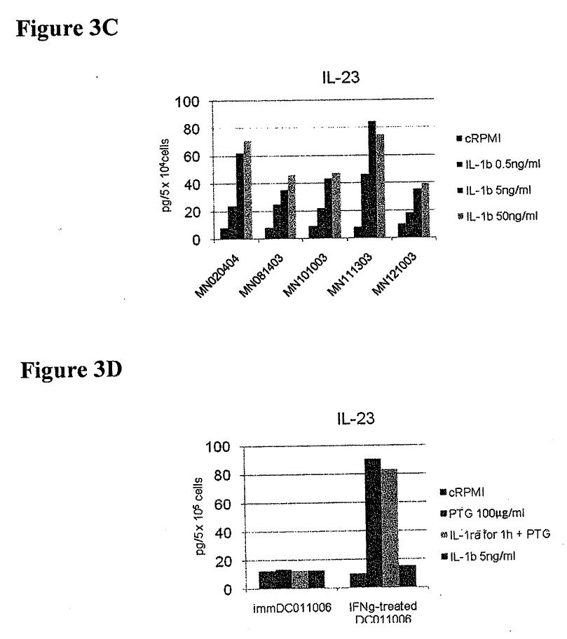

To better understand the contribution of IL-1 signaling in the IL-23/T_{H}17 innate response to PTG in monocyte and immDC populations, we pretreated monocytes, immDC and IFNγ-treated immDC with the naturally occurring anti-inflammatory IL-1 receptor antagonist (IL-1ra) for 1 h prior to stimulation with PTG or β-glucan as a positive control, or incubated with increasing doses of exogenous IL-1β for 20 h. Addition of IL-1ra drastically inhibited the IL-23, TNFα, IL-6, and CCL20 responses to PTG in monocytes, demonstrating the influential role of autocrine IL-1 signaling in monocyte-derived IL-23/T_{H}17 innate immune responses (FIG. 3A). The requirement for IL-1 appears to be a general phenomenon of this pathway in monocytes, since IL-1ra also inhibited the cytokine response to the positive control β-glucan (FIG. 3B), and the IL-23 response to exogenous IL-1β was dose dependent (FIG. 3C). Similar effects were observed for the other cytokines/chemokines. Contradictory to the monocyte response, addition of IL-1ra or IL-1β to IFNγ-treated immDC had no effect on the IL-23/T_{H}17 profile induced by PTG exposure (FIG. 3D), providing further indication that monocytes exposed to gluten and not their progeny DC have the potential to activate autoreactive memory T_{H}17 cells. Indeed, other groups have reported in human studies that activated monocytes, which produce IL-1β and IL-23, are the best inducers of T_{H}17 cells (Evans et al., Proc. Natl. Acad. Sci., U.S.A., 104(43):17034-17039 (2007)).

Subtypes of Monocytes Required for the IL-1β and IL-23 Response to PTG

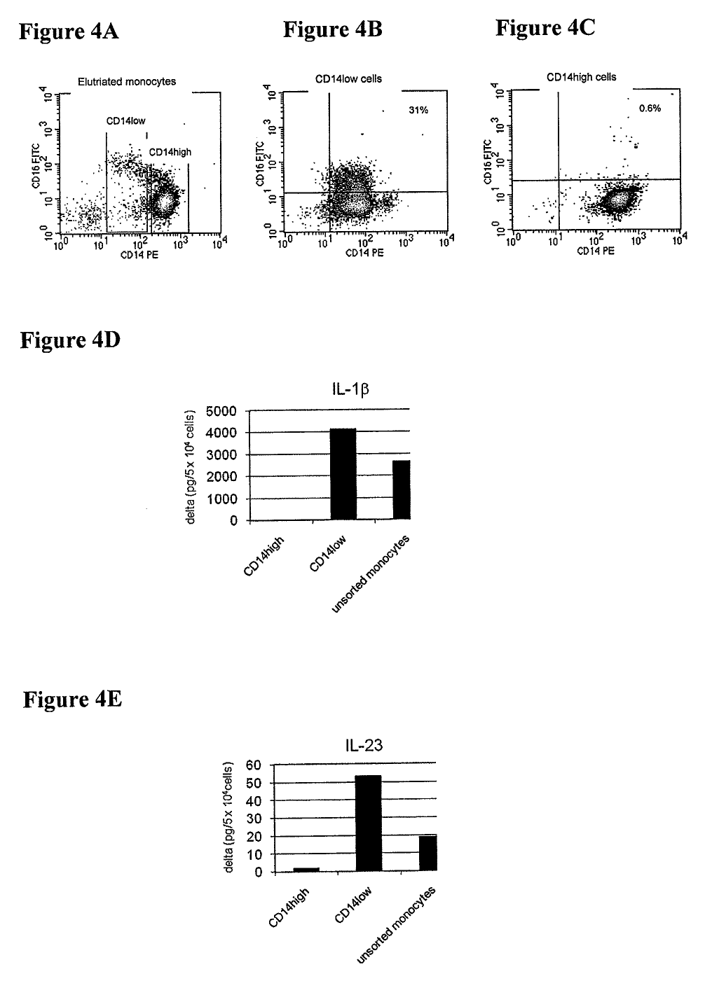

The minor CD14^{low}CD16⁺ subtype of monocytes has a greater capacity to produce TNFα, IL-1β and IL-6, and is expanded in conditions of chronic inflammation such as autoimmune disease. Given the proinflammatory nature of this subset, and the similar cytokine profile triggered by PTG in monocytes, we investigated the involvement of CD14^{low}CD16⁺ cells in our in vitro model of the IL-23/T_{H}17 innate immune response to PTG. Elutriated monocytes were stained with αCD14-PE mAb and sterile sorted based on high and low expression of CD14 since the majority of CD16⁺ cells are CD14^{low} and αCD16 monoclonal antibody reportedly alters their function (FIGS. 4A-4E). Equal numbers of CD14^{high}, CD14^{low} or unsorted control monocytes were incubated in the presence or absence of 100 ug/ml PTG for 20 h, and cell free supernatants were analyzed for IL-1β and IL-23 (ELISA). Remarkably, only CD14^{low}CD16⁺ monocytes produced IL-1β and IL-23 in response to stimulation with PTG, providing a more specific target population that can be exploited for further investigation of the receptors and signaling mechanisms involved (FIGS. 4A-4E).

Compare the proportion of populations of monocytes, T cells, and their respective subsets associated with the IL-23/T_{H}17 pathway in PBMC from patients with active and treated CD and HLA-DQ matched and HLA DQ mismatched healthy individuals.

Increased CD14^{low}CD16⁺ Monocytes in CD Patients

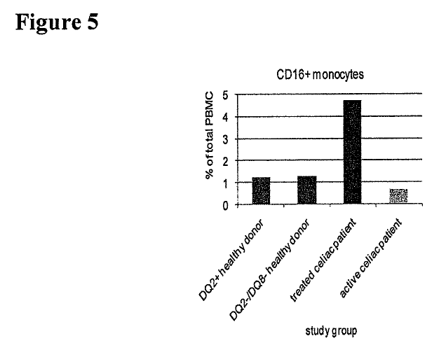

Given that this subset is expanded in other autoimmune diseases and has been observed in active celiac lesions, together with our novel discovery that this subset is required for the IL-23/T_{H}17 innate immune response, we further investigated the proportion of this subset. Freshly thawed PBMC from CD patients with active and treated disease and HLA matched and mismatched normals were stained with monoclonal Abs to CD11c, CD14 and CD16, or appropriate isotype controls and analyzed by flow cytometry. The proportion of CD14^{low}CD16⁺ monocytes was considerably highest in the CD patient with treated disease and lowest in the CD patient with active disease (FIG. 5), which can be explained by the increase of this subset in the intestinal tissues of CD patients exposed to gluten (Raki et al., Gastroenterology, 131(2):428-438 (2006)).

Effect of gliadin on PBMC from treated and active CD patients and HLA-matched and mismatched healthy donors.

Influence of HLA class II Alleles on Gliadin-Induced T_{H}17 Related Cytokines/Chemokines

Given the HLA requirement for CD, together with the knowledge that gliadin activates the proinflammatory cytokines TNFα and IL-8 in PBMC from healthy individuals, we evaluated its capacity to stimulate T_{H}17 related cytokines in HLA-matched and mismatched healthy subjects.

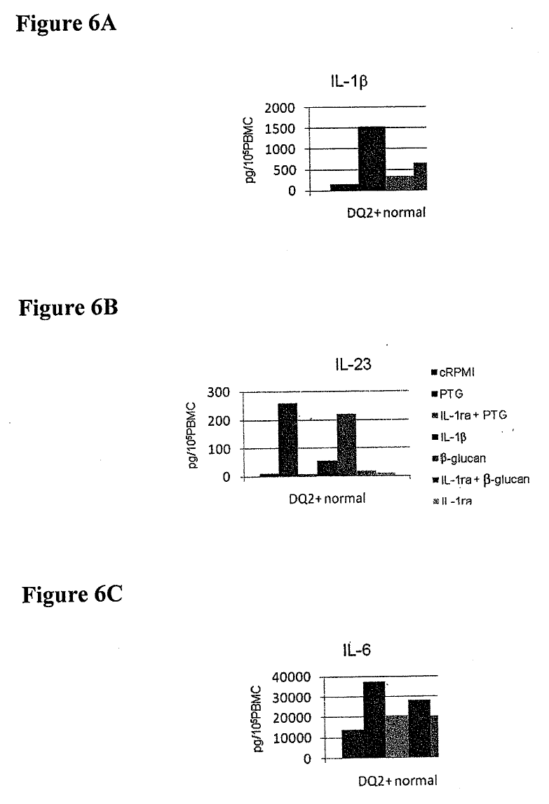

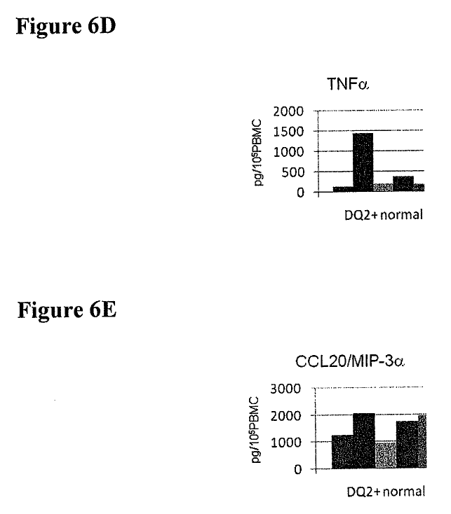

We cultured PBMC from HLA-DQ2 positive and HLA-DQ2 negative healthy persons in the presence or absence of 100 ug/ml of PTG or β-D-glucan from barley as a positive control for 20 h. Cell-free culture supernatants were harvested and analyzed for proinflammatory cytokines IL-1β, IL-6, IFNγ, TNFα (Luminex) IL-23 and the T_{H}17 chemokine CCL20/MIP-3α (ELISA). PTG stimulated production of IL-23, IL-1β, IL-6, TNFα and CCL20, and not the IL-17 inhibitor IFNγ in all donors tested, illustrating the ability of PTG to induce the IL-23/T_{H}17 innate immune response (FIGS. 6A-6E).

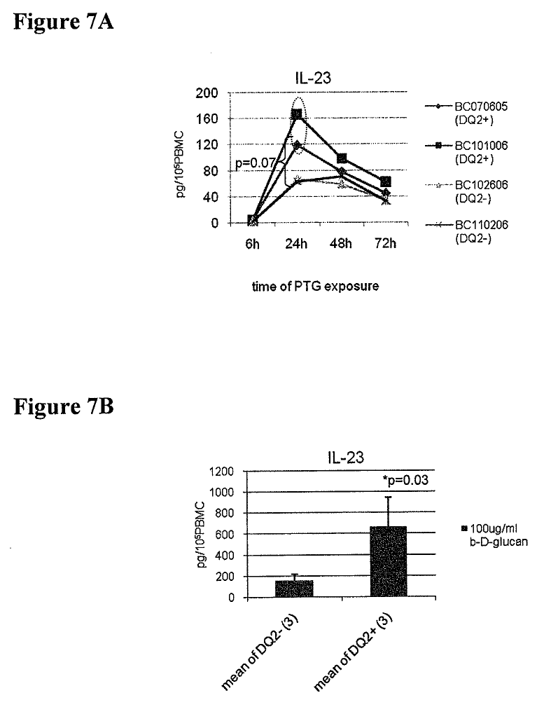

Interestingly, PTG and β-glucan had a more pronounced effect on PBMC from HLA-DQ2⁺ healthy individuals, suggesting a positive correlation between the HLA-DQ alleles required for CD and the intensity of the IL-23/T_{H}17 innate immune response (FIGS. 7A-7B). Kinetic studies evaluated after 6, 24, 48 and 72 h exposure to PTG revealed secretion of IL-1β, IL-6 and TNFα as early as 6 h, while IL-23 was not detected until 24 h at which time it reached peak levels and declined steadily thereafter (FIGS. 7A-7B). Considering the essential role for IL-1β in the IL-23/T_{H}17 innate response in purified monocytes, we predicted that IL-1β regulates the cytokine profile triggered by PTG and β-glucan in whole PBMC as well. To this end, PBMC were pretreated with IL-1ra for 1 h prior to stimulation with PTG or β-glucan, or incubated with 5 ng/ml IL-1β alone for 20 h. Indeed, IL-1ra almost completely inhibited IL-23, TNF-α, IL-6, and CCL20 responses to PTG and β-glucan, implying that IL-1 signaling was required for production of these potent mediators. IL-1ra also reduced the IL-1β response by ˜60%, indicating a positive feedback loop wherein the initial burst of IL-1β released upon engagement of PTG with its anonymous PRR perpetuates IL-1β secretion. Moreover, exogenous IL-1β generated a similar profile as PTG producing equivalent concentrations of CCL20 and IL-6, but reduced levels of IL-23 and TNFα suggesting that the complex cytokine milieu triggered by PTG enhance the IL-23 and TNFα responses (FIGS. 6A-6E).

The Cytokine Response to Gliadin is Augmented in CD Patients on GFD Compared to HLA-DQ2 Matched Healthy Individuals

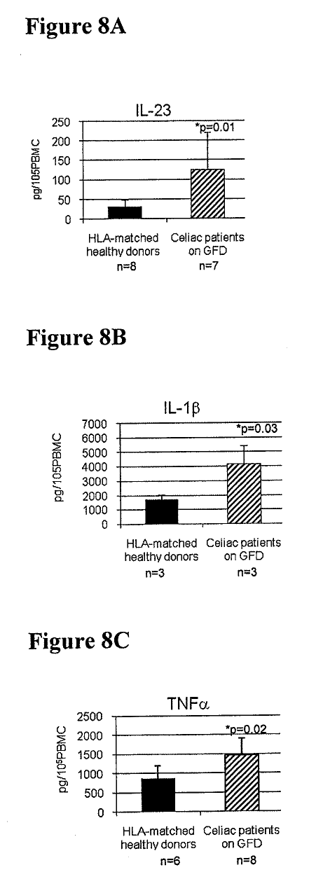

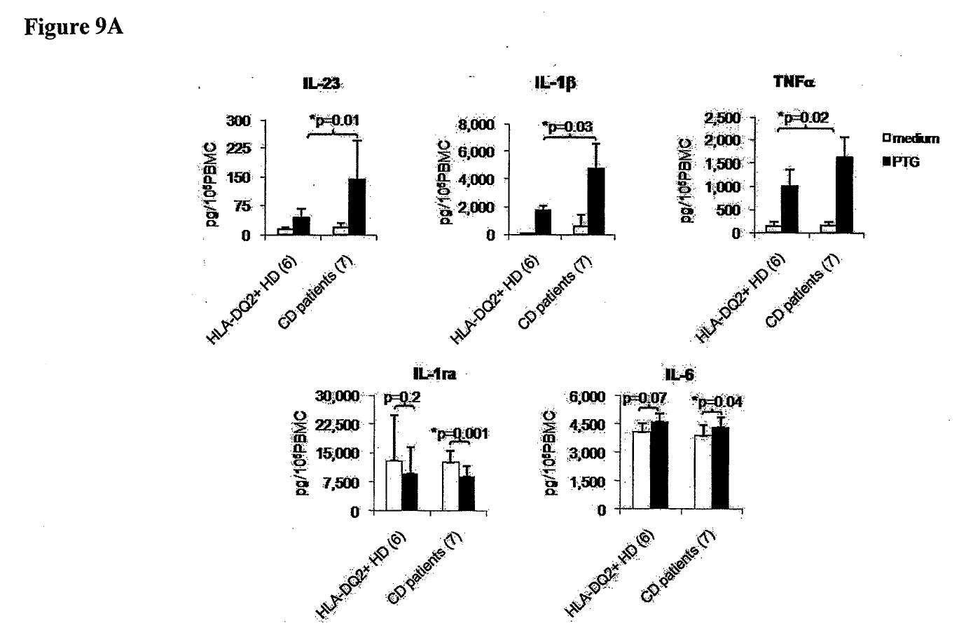

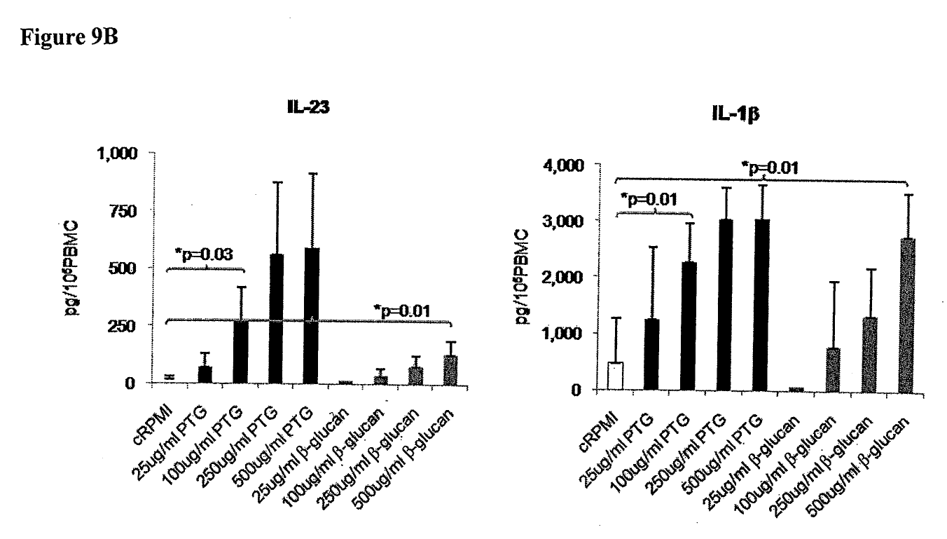

Considering the role of the IL-23/T_{H}17 pathway in autoimmune diseases sharing the disease-associated HLA class II alleles, and our novel discovery that PTG stimulates this innate immune response in PBMC of healthy subjects, we tested the hypothesis that PTG activates the IL-23/T_{H}17 pathway differently in individuals with CD compared to healthy individuals. We incubated PBMC from CD patients on a GFD in remission and PBMC from HLA-matched healthy donors in the presence or absence of PTG, and quantified levels of IL-1β, IFNγ, TNFα (Luminex) and IL-23 (ELISA) from cell-free culture supernatants after 48 h. Significantly elevated levels of IL-23, IL-1β and TNFα were detected in culture supernatants from PBMC from the CD patients, indicating a role for the IL-23/T_{H}17 paradigm in the pathogenesis of CD (FIGS. 8A-8C). A correlation between the proportion and activation status of cell subsets involved in the IL-23/T_{H}17 axis and the differential cytokine response to PTG was observed.

We discovered the involvement of IL23-mediated inflammation in the pathogenesis of CD and address a number of unresolved questions pertinent to the long felt need, such as: Is there a role for HLA-DQ2 (and/or -DQ8) or linked genes in the activation of this innate immune response? What is responsible for the difference in the response observed in CD patients vs. healthy subjects with the same HLA genotype? Does this innate immune response control the balance between chronic inflammation and tolerance to a food stuff that is consumed by the population at large, but causes disease in those genetically predisposed? What is the constituent(s) in gliadin that initiates the production of cytokines and chemokines that characterize the IL-23/T_{H}17 response and what PRR on APC interacts with this constituent? What genes are expressed as a result of the PRR-ligand interaction?

### EXAMPLES

We used several approaches to test the overall hypothesis that the IL-23/T_{H}17 innate immune response axis is activated by exposure of defined populations of APC to gliadin proteins, and that this innate immune response plays a fundamental role in the pathogenesis of CD in genetically predisposed individuals.

### Example 1

We identified the subset of monocytes and/or immature DC that produce IL-23 and related T_{H}17 cytokines in response to PTG exposure. Highly purified monocytes from our inventory of healthy individuals will be used to investigate this AIM. Elutriated monocytes are obtained from the apheresis products of healthy individuals and cryopreserved until used. Freshly thawed monocytes will be sorted into CD14^{hi} and CD14^{low} subsets or cultured with GM-CSF and IL-4 to make immDC that will be incubated for an additional 18 h in the presence or absence of IFNγ. Unsorted monocytes, monocyte subsets, immDC and IFNγ-treated immDC will be exposed to PTG with and without IL-1ra, β-glucan or IL-1β for 20 h. Cell-free culture supernatants will be harvested and analyzed for cytokine/chemokine production by ELISA.

Isolation of subsets of monocytes: Subsets of monocytes will be separated by sterile flow cytometry cell sorting. Cells are incubated with fluorochrome-conjugated CD14 antibody for 15 minutes at 27 C, washed and resuspended in FACS buffer (PBS with 1% huAB serum) and sorted in CD14^{high}, CD14^{low} and CD14⁻ subsets on a BD FACSVantage cell sorter.

Antigens: Gliadin will be prepared by enzymatic digestion as described previously (Thomas et al., J. Immunol., 176(4):2512-2521 (2006)). Briefly, 50 g of gliadin (crude wheat; Sigma-Aldrich) is dissolved in 500 ml of 0.2 N HCl for 2 h at 37° C. with 1 g of pepsin (Sigma-Aldrich). The resultant peptic digest is further digested by the addition of 1 g of trypsin (Sigma-Aldrich) after the pH was adjusted to 7.4 using 2 M NaOH. The solution is stirred vigorously at 37° C. for 4 h, boiled (100° C.) for 30 min, freeze-dried, lyophilized in 10-mg aliquots, and stored at −20° C. until use (referred to as pepsin/trypsin-digested gliadin or (PTG). 100 mg of β-D-glucan from barley (Sigma-Aldrich) is dissolved in 600 ul 95% EtOH followed by 9 mL distilled water. The resultant slurry is then stirred vigorously at 100° C. for 3 min. or until completely dissolved, allowed to cool, and stored at 10 mg/ml at 4° C. until used.

ELISA Cytokine Assays: Cytokine levels are quantified using IL-23 (eBioscience) or Quantikine ELISA kits (R& D Systems) following the manufacturers' protocol. Briefly, samples and standards (100 ul) are added to each well in duplicate and incubated at room temperature (RT) for 2 hrs. Wells are washed four times with wash buffer. Conjugate is added to each well and incubated for 1-2 hrs at RT. After washing four times, substrate solution is added to each well and the plate is incubated for 15-30 min at RT. The reaction is stopped by adding 50 ul of stop solution to each well and the OD 450 nm read.

The CD14^{low}(CD16⁺) monocyte population generate an IL-1 dependent IL-23/T_{H}17 innate response with exposure to PTG, β-glucan and IL-1β. IFNγ-treated immDC will produce IL-23 and related cytokines (except for IL-1β) independently of IL-1β in response to PTG, and immDC not exposed to IFNγ will not recapitulate the IL-23/T_{H}17 response to these antigens.

Variables introduced by genetic diversity, lifestyle and environment, blood sample collection and processing, and the cell sorting process could impact the phenotype and function of monocytes in vitro. Monocyte subsets are magnetically depleted from whole PBMC or elutriated monocytes, and the untouched cells tested for reactivity to PTG and β-glucan. Intracellular cytokine staining is used to identify the subsets producing IL-1β in response to antigens, since an appropriate antibody for detecting IL-23 is not currently available.

### Example 2

We identified, enumerated and compared the proportion of monocytes that produce IL-23 in response to PTG and the number of T_{H}17 cells in PBMC obtained from patients with active and treated CD and HLA-DQ matched and HLA-DQ mismatched normal individuals.

Peripheral blood samples are obtained from patients. Disease, treatment status as well as relationship to other donors are recorded. Peripheral blood samples are obtained from patients with untreated disease, treated (on a gluten-free diet), HLA-DQ2/DQ8⁺ and HLA-DQ2/8⁻ individuals that are disease free. The laboratory investigators are blinded to this information until after completion of the following tests. Serum samples from all study subjects are tested for antibodies to tissue transglutaminase (tTG). PBMC are isolated from whole blood, for example by density gradient centrifugation, and DNA extracted from a portion of the cells for high resolution HLA class II allele typing (HLA-DRB1, DQA1 and DQB1) and future molecular studies. The remainder and the majority of the PBMC will be cryo-preserved by control rate freezing and stored in the vapor phase of liquid nitrogen until used.

PBMC Cryopreservation: Peripheral blood mononuclear cells are isolated from donors' whole blood, for example by density gradient centrifugation in Lymphocyte Separation Medium (ICN Biomedicals Inc., Aurora, Ohio). These cells will be viably cryopreserved, for example in RPMI-1640 media (Invitrogen Corp., Grand Island, N.Y.) containing 20% human AB serum (Gemini Bioproducts, Woodland, Calif.) and 10% Dimethylsulfoxide (Sigma, St Louis, Mo.) or other suitable media using an automated cell freezer (Gordinier Electronics, Roseville, Mich.) and stored in the vapor phase of liquid nitrogen until used.

Combinations of fluorochrome-conjugated antibodies and flow cytometric technology are used to identify cells and subsets of cells of the various lineages in PBMC obtained from patients and controls. To identify and enumerate B cells, suitable targets include CD19, CD27; NK cells, suitable targets include CD3, CD56; T cells and T cell subsets, suitable targets include CD3, CD4, CD8, CD56, αβTCR, γδTCR, CD45RO, CD45RA, IL1R, IL23R, CCR6, CXCR3 and activation status suitable targets include CD25 and CD69; monocytes/dendrites cells suitable targets include CD11c, CD1a, CD14, CD16, CD32, CD64, CD123, HLA-DR, HLA-DQ, HLA-DP, CD40, CD80, CD86, CD83, TLR2, CX3CR1, CCR2, GM1, CCR6. Data is analyzed comparing the relative distribution of different cell lineages and lineage subsets. While the analysis is focused on the differences in the numbers of T_{H}17 cells (CD4⁺CD45RO⁺IL23R⁺CCR6⁺IL1R⁺) and the CD16⁺CD14⁺ monocyte subset in the patients and controls, this systematic comparison of the distribution of mononuclear cells provides a baseline for functional studies.

Flow Cytometry: Cells are labeled using flow cytometry methodology. Briefly, cells are washed in RPMI containing 5% hAB, and incubated for 15 minutes at room temperature (22-25° C.) in order to block Fc receptors. After an additional wash, (1×PBS with 1% FBS and 0.1% NaN₃) fluorochrome-conjugated antibodies are added (BD Biosciences, San Diego, Calif.) and the cells incubated for 30 minutes at 4° C., washed again and fixed in 1×PBC with 1% paraformaldehyde. Flow cytometric data are acquired using a BD™ FACS SCAN flow cytometer and analyzed with CellQuest Software (Becton Dickinson, San Jose, Calif.).

We see a significant difference in the relative numbers of circulating cells that express the combinations of markers that identify the monocyte population that produces IL-23 and other T_{H}17 related cytokines in response to PTG. The highest number of will be found in treated celiac patients, with lower numbers in patients among other groups. Our model hypothesizes that the monocyte population that produces IL-23 and other T_{H}17 related cytokines in response to PTG localize in the gut and are particularly abundant in patients with active disease thus lower numbers in circulation. In studies determining the phenotype of the T cell populations in the four groups, we see the highest numbers of the CD4⁺ cells with the T_{H}17 phenotype (CD45RO, IL23R, CCR6) in CD patients with active disease and these numbers lowered as the patients are followed after a change in diet to reduce or eliminate gliadins.

Phenotyping of intestinal tissue from patient populations is done using an inventory of paraffin-embedded intestinal biopsies from an Amish cohort of related individuals with and without CD that will be available for study.

### Example 3

The magnitude and array of cytokine/chemokine responses to PTG is different in active and treated CD patients from that observed in PBMC from HLA-DQ matched and HLA-DQ mismatched normal individuals.

PBMC from patients and controls are exposed to PTG, IL-1β, IL-1 receptor antagonist, and β-glucan for 6, 24, 48 and 72 hours, and the culture supernatants are harvested for cytokine analysis. The cell-free culture supernatants are assayed for the presence and quantity of cytokines, such as IL-1, IL-23, IL-21, IFNγ, TNFα, IL-6, IL-8, IP-10, IL-2, IL-10, IL-4, MIP3α, IL-17 (A-F) and IL-27 in ELISA assays or alternatively in multiplex cytokine/chemokine assays. These experiments are initially on PBMC from subjects in different groups to determine if the kinetics of response is similar or different. Using the information obtained from these kinetic studies, we select the conditions and the length of culture that provides optimal information when PBMC from other individuals in each of the groups are tested. The rational for selecting IL-1β and its natural inhibitor comes from our observation that production of this cytokine precedes production of IL-23, and that the presence of IL-1ra abrogates the IL-23 response induced by PTG. β-glucans activate the IL-23/T_{H}17 innate immune response. Inclusion of IL-23 is a positive control as our results indicate that the response of PBMC to β-glucan exposure is similar to the response observed when the same cells are exposed to PTG.

Antigens and ELISA Methods

Bio-Plex Human Cytokine Assay: Cytokines from cell culture supernatants are detected and quantified using various commercially available kits and reagents, including Bio-Plex Cytokine Assay kits (Bio-Rad). Briefly, a premixed standard is reconstituted in the same culture media as that used for the sample and incubated on ice for 30 minutes. Serial dilutions of the stock are prepared to give a total of eight standards. Cell culture supernatants are diluted and kept on ice until ready to use. Premixed beads (50 ul) coated with target capture antibodies are transferred to each well of a filter plate and washed twice with buffer. The premixed standards and samples (50 ul) are added to each well. The plate is shaken for 30 sec. and incubated for 30 minutes at RT with shaking at 300 rpm. After washing, premixed detection antibodies (50 ul) are added to each well and shaken at RT for an additional 10 minutes. After 3 washes, the beads are resuspended in 125 ul of Bio-Plex assay buffer. The plate is then read on the Bio-Plex suspension array system and analyzed using Bio-Plex Manager software.

Having identified the cytokines/chemokines produced by PBMC from the different cohorts, studies are conducted to determine the population of cells producing the lymphokines. PBMC are exposed to PTG, IL-1β and β-glucan in culture for the period of time that was determined to produce the peak amounts of the cytokines/chemokines and the cells producing the cytokine and/or chemokines identified using intracellular staining and flow cytometric methods.

Detection of intracellular cytokine expression by flow cytometry: Intracellular cytokine production by sub-populations of PBMC is measured by flow cytometry. For example, cells are stimulated with the selected antigen (or mitogen) for 2 hours at 37° C., 5% CO₂ in a humidified incubator. Golgi Stop (10 μg/ml) (Pharmingen, San Diego, Calif.) is added for an additional 4 hours of incubation. Cells are washed with blocking buffer (PBS with 5% human AB serum), resuspended in the buffer and incubated for 15 minutes at 4° C. to block Fc receptors. The cells are washed with staining buffer (PBS with 1% human AB serum) and incubated with fluorochrome labeled antibodies detecting cell surface markers or appropriate isotype controls (Becton Dickinson, San Jose, Calif.) at 4° C. for 25 minutes in the dark. After washing, cells are fixed and permeabilized by incubation with 250 ul perm/fix solution (Pharmingen, San Diego, Calif.) for 25 minutes at 4° C., then washed with perm wash buffer. The fluorochrome labeled monoclonal for intracellular cytokines or appropriate isotype control antibodies are then added (Pharmingen, San Diego, Calif.) and incubated at 4° C. for 25 minutes in the dark. The cells are again washed, resuspended in 200 ul perm wash buffer and analyzed by flow cytometry.

The array and magnitude of cytokines/chemokines differ in PBMC from CD patients with active disease, patients on a disease free diet and controls. We see differences depending on HLA class II type in control populations. The levels of cytokine/chemokine production in response to PTG exposure corresponds to the numbers of CD16⁺ monocytes. The responses observed will be replicated when PBMC are exposed to β-glucans and addition of IL-1ra shuts down production of all of the cytokines involved in the innate immune response regardless of the agent that was used to induce the response. A central role for IL-1β or its receptor in the initiation of the IL-23/T_{H}17 pathway is established, thus therapeutic options are available.

### Example 4

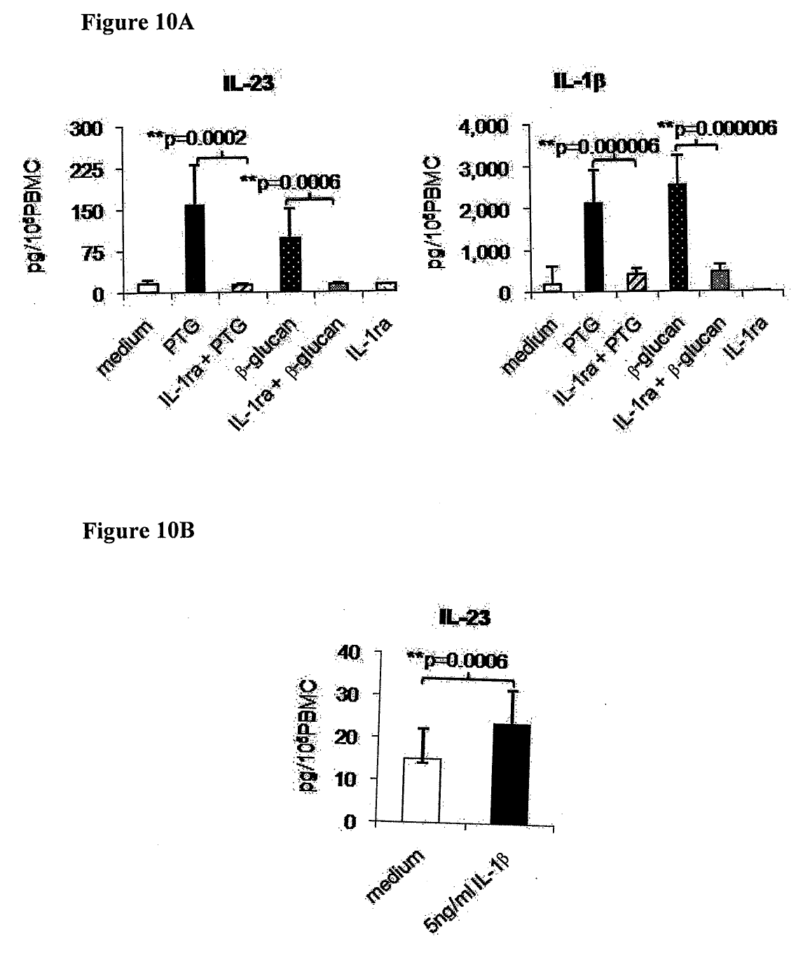

The kinetics of cytokine responses to PTG was determined by exposing PBMC to PTG for 6, 24, 48 and 72 h. These studies revealed that IL-1β, IL-6 and TNFα were secreted in as few as 6 h following PTG exposure, while IL-23 could not be detected until 24 h, suggesting that induction of IL-23 required earlier inflammatory mediators (data not shown). During these initial studies, we also observed a positive correlation between IL-1β and IL-23, which led us to hypothesize that IL-1 is essential for production of IL-23. To directly examine the role of IL-1β in IL-23 responses, we treated PBMC from CD patients with IL-1ra prior to stimulation with PTG or the positive control, β-glucan. IL-1ra completely inhibited induction of IL-23 in response to both PTG and β-glucan, illustrating the fundamental role of IL-1 signaling in IL-23 production (FIG. 10A). IL-1ra also markedly reduced levels of IL-1β in PBMC treated with both antigens, suggesting that IL-1β released upon engagement of PTG or β-glucan with their respective pattern recognition receptor (PRR) perpetuates production of IL-1β and facilitates induction of IL-23 (FIG. 10B). Additionally, PBMC were treated with physiologic concentrations of exogenous IL-1β in order to ascertain its direct effects on cytokine production. Importantly, IL-1β alone induced IL-23 production at much lower levels than PTG and β-glucan, indicating that additional signaling pathways triggered by these antigens enhance secretion of IL-23 (FIG. 10B). The invention demonstrates for the first time that the IL-1 system regulates IL-23, and illustrate the powerful anti-inflammatory effects of IL-1ra on induction of IL-23.

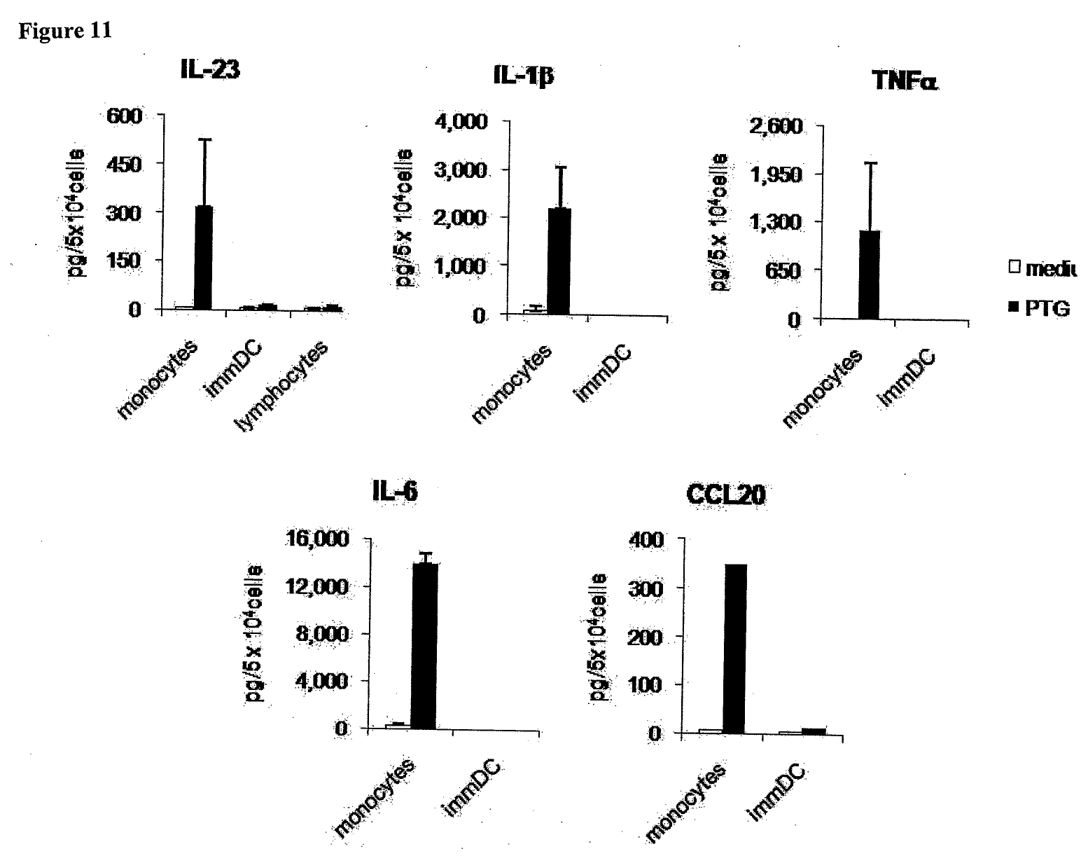

While IL-1β is produced by many cell types, IL-23 production is thought to be restricted to activated APC. Recently, TLR activated monocytes were shown to secrete high levels of IL-23 and to be the best inducers of Th17 cells¹⁴, thus we predicted that monocytes were the cellular source of PTG-induced IL-23. To investigate this hypothesis, we exposed purified lymphocytes, monocytes, or monocyte-derived DC (cultured with GM-CSF and IL-4 for 72 h) to PTG overnight and analyzed the cell-free culture supernatants for IL-23 and related “Th17” polarizing mediators. Under these conditions, monocytes and not their progeny DC or lymphocytes produced IL-23, IL-1β, IL-6, TNFα and CCL20 in response to PTG, demonstrating a direct interaction between PTG and its anonymous PRR(s) on this population (FIG. 11).

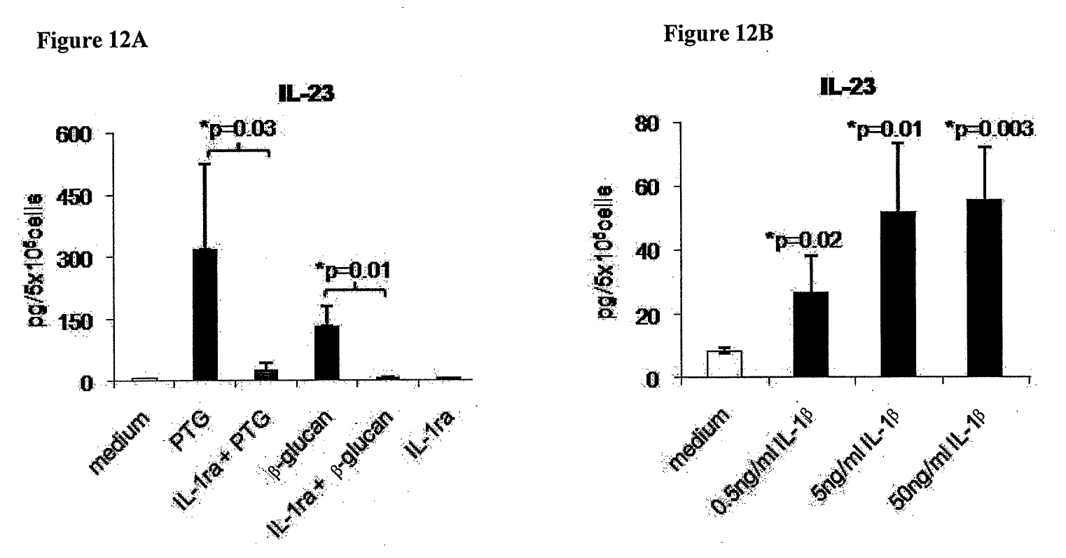

As with whole PBMC, IL-1ra significantly inhibited IL-23 responses to PTG and β-glucan in purified monocytes (FIG. 12A), and addition of exogenous IL-1β to this subset triggered a dose-dependent IL-23 response (FIG. 12B). These results illustrate that gliadin directly stimulates monocytes to secrete IL-23 and related inflammatory mediators and further support a primary role for the IL-1 system in IL-23 mediated inflammation.

In summary, our studies demonstrate that enzymatically digested wheat gliadin stimulates monocytes to produce significantly more IL-23, IL-1β and TNFα in CD patients than HLA-DQ2⁺ healthy individuals, and reveal a fundamental role for the IL-1 system in the IL-23 pathway. We show that IL-1β directly induces monocytes to secrete IL-23, while its natural inhibitor, IL-1ra, substantially inhibits both the IL-1β and IL-23 responses generated by monocytes exposed to gliadin. Moreover, our data indicate that gliadin initiates the inflammatory cascade by disrupting the balance between these two IL-1 members, which could be targeted therapeutically for treatment of this disease and other conditions associated with IL-23 mediated inflammation.

### Methods

Cells. Peripheral blood mononuclear cells (PBMC) were isolated from Celiac patients' and healthy donors' whole blood by methods common in the art including, for example, by density gradient centrifugation in Lymphocyte Separation Medium (ICN Biomedicals Inc.). PBMC were viably cryopreserved in RPMI-1640 media (Invitrogen Corp.) containing 20% human AB serum (hAB) (Gemini Bioproducts) and 10% Dimethylsulfoxide (Sigma) using an automated cell freezer (Gordinier Electronics), and stored in the vapor phase of liquid nitrogen until used. Highly purified monocytes (95% purity) were obtained from healthy donors as above followed by countercurrent centrifugal elutriation. The resulting cells were viably cryopreserved in fetal bovine serum (Summit Biotechnology) containing 10% DMSO and 5% glucose (Sigma) for later use. All individuals gave informed consent for peripheral blood drawn for this study. The study protocol was approved by the Institutional Review Board at the University of Maryland School of Medicine.

DNA Extraction and HLA Typing. DNA was extracted using common methods and reagents, for example from a portion of the PBMC using the QIAamp DNA Mini Kit (Qiagen) per the manufacturer's instructions. DNA was analyzed by spectrophotometry to determine quantity and purity and stored at −20° C. until used. Alleles of genes encoding HLA were identified using One Lambda Micro SSP™ ABDR Typing Kit, and alleles of genes encoding HLA-DQ were determined by DQA1 and DQB1 SSP UniTray® Kit (Dynal Biotech) following the manufacturer's.

Reagents. Gliadin was prepared by enzymatic digestion as described previously (Thomas et al., J. Immunol., 176:2512-2521 (2006)). The presence of contaminating endotoxin in gliadin was determined by Limulus amebocyte assay per the manufacturers' instructions. 100 mg of β-D-glucan from barley (Sigma) was dissolved in 600 ul 95% EtOH followed by 9 mL distilled water. The resultant slurry was stirred vigorously at 100° C. for 3 minutes, allowed to cool, and stored at 10 mg/ml at 4° C. until used. 25 overlapping 20 mers spanning the sequence of α-gliadin were synthesized and purified >95% at the University of Maryland Biopolymer Lab, and stored at −20° C. until used. Recombinant human IL-1β and IL-1ra were purchased from R & D Systems.

PBMC cultures. PBMC from CD patients and HLA-DQ2⁺ healthy controls was tested as follows. 10⁶ PBMC/ml were incubated in RPMI-1640 supplemented with 10% heat inactivated hAB, 1% L-glutamine, 1% Pen-Strep and 20 mM Hepes Buffer (cRPMI) with and without PTG, β-glucan, 5 ng/ml rhIL-1β, or 10 μg/ml pooled synthetic 20 mers of α-gliadin in 96 well U-bottom plates (Denville Scientific Inc.) at 37° C. in 5% CO₂ for 6, 24, 48, or 72 h. Alternatively, 10⁶ PBMC/ml were incubated with 0.5 μg/ml rhIL-1ra at 37° C. in 5% CO₂ for 1 h then cultured with and without 100 μg/ml PTG or 500 μg/ml β-glucan for an additional 20 h. Cell-free culture supernatants were harvested for cytokine and chemokine analysis.

Elutriated monocyte cultures. 5×10⁵ monocytes/ml were cultured in cRPMI with and without 100 μg/ml PTG, 100 μg/ml β-glucan, or 0.5-50 ng/ml rhIL-1β in 96 well U-bottom plates at 37° C. in 5% CO₂ for 20 h. Alternatively, 5×10⁵ monocytes/ml were incubated with 0.5 μg/ml rhIL-1ra at 37° C. in 5% CO₂ for 1 h then cultured with and without 100 μg/ml PTG or β-glucan for an additional 20 h. Cell-free culture supernatants were harvested for cytokine and chemokine analysis.

Cytokine & chemokine analysis. Cell-free culture supernatants were analyzed for IL-1β, IL-1ra, IL-6, IL-12p70, IFNγ, TNFα (Bio-Plex Cytokine Assay kit, Bio-Rad) or IL-1β, IL-23 (ELISA kit, eBiosciences), IL-1ra and CCL20 (Quantikine ELISA kit, R & D Systems) following the manufacturers' protocols. Appropriate standard curves were included in each assay.

Statistical analyses. Data are presented as mean values+s.d. P values comparing different conditions within the same individuals were calculated using paired two-tailed Student's t tests and p values comparing the two study groups were determined by unpaired two-tailed Student's t tests (FIG. 1A). P values<0.05 were considered statistically significant.

Certain patents and printed publications have been referred to in the present disclosure, the teachings of which are hereby each incorporated in their respective entireties by reference.

While the invention has been described in detail and with reference to specific objects, examples or embodiments thereof, it will be apparent to one of ordinary skill in the art that various changes and modifications can be made thereto without departing from the spirit and scope of the invention.

What is claimed:

1. A method of reducing inflammation in a subject with celiac disease comprising administering to said subject an IL-1 antagonist or an IL-1 inhibitor wherein said administering of said IL-1 antagonist or said IL-1 inhibitor reduces inflammation in said subject with celiac disease.

2. The method of claim 1, wherein said inflammation is IL-23 mediated inflammation.

3. The method of claim 1, wherein said administering of an IL-1 antagonist or an IL-1 inhibitor causes a reduction of IL-23 or IL-1β.

4. The method of claim 1, wherein said administering of an IL-1 antagonist or an IL-1 inhibitor causes a reduction of IL-23.

5. The method of claim 1, wherein said IL-1 antagonist or said IL-1 inhibitor is IL-1ra.

6. A method of influencing immunity in an individual with celiac disease.

7. The method of claim 6, comprising influencing IL-23.

8. The method of claim 6, comprising influencing T_{H}17 cytokines.

9. The method of claim 6, comprising influencing innate immunity.

10. A diagnostic method, comprising contacting at least one cell wherein said cell is obtained from an individual with celiac disease, further comprising contacting at least one cell wherein said cell is obtained from an individual without celiac disease, further comprising comparing said cell from an individual with celiac disease to said cell from an individual without celiac disease, and further comprising comparing IL-1β from an individual without celiac disease to IL-1β from an individual with celiac disease.

11. A diagnostic method, comprising contacting at least one cell wherein said cell is obtained from an individual with celiac disease, further comprising contacting at least one cell wherein said cell is obtained from an individual without celiac disease, further comprising comparing said cell from an individual with celiac disease to said cell from an individual without celiac disease, and further comprising comparing IL-1RA from an individual without celiac disease to IL-1RA from an individual with celiac disease.


SEQUENCE LISTING (4 sequences)

SEQ ID NO: 1 - protein, 7 aa, Homo sapiens
RYTVELA

SEQ ID NO: 2 - protein, 9 aa, Homo sapiens
MKLPVHKLY

SEQ ID NO: 3 - protein, 11 aa, Homo sapiens
VGSPKNAVPPV

SEQ ID NO: 4 - protein, 11 aa, Homo sapiens
WTLDGKKPDDL